(19) United States
(12) Patent Application Publication
(10) Pub. No.: US 2022/0354816 A1
(43) Pub. Date: Nov. 10, 2022

(54) OSELTAMIVIR FORMULATION

(71) Applicant: SUNSHINE LAKE PHARMA CO., LTD., Dongguan, Guangdong (CN)

(72) Inventors: Xue LI, Dongguan (CN); Xin HUANG, Dongguan (CN); Jinsong YOU, Dongguan (CN); Fangfang HUANG, Dongguan (CN)

(73) Assignee: SUNSHINE LAKE PHARMA CO., LTD., Dongguan, Guangdong (CN)

(21) Appl. No.: 17/764,322

(22) PCT Filed: Sep. 25, 2020
(86) PCT No.: PCT/CN2020/117625

§ 371 Date: Mar. 28, 2022

(30) Foreign Application Priority Data
Sep. 27, 2019 (CN) 201910926421.X
Nov. 28, 2019 (CN) 201911187613.X
Jun. 4, 2020 (CN) 202010501774.8

(51) Int. Cl. A61K 31/215; A61K 9/16; A61K 9/20

(52) U.S. Cl. CPC A61K 31/215; A61K 9/1652; A61K 9/2086; A61K 9/1635

(57) ABSTRACT

An oseltamivir formulation and a preparation method of the formulation, the method being simple to operate, having good reproducibility, and being suitable for manufacture. The oseltamivir formulation includes oseltamivir or a salt thereof and a sustained-release material. The formulation may be a single-phase release formulation, a dual-phase release formulation, a three-phase release formulation, or a multi-phase release formulation having more than three phases. The formulation is administered once-daily and can achieve sustained release of at least 24 hours or longer, which can reduce the times of administration and avoid peak-to-valley fluctuations, thereby improving the compliance and safety of patients.

### FIELD OF THE INVENTION

The invention relates to the field of pharmaceutical formulations, in particular to a formulation of oseltamivir or salt thereof.

### BACKGROUND

The molecular formula of oseltamivir is C₁₆H₂₈N₂O₄, and its chemical name is ethyl (3R,4R, 5S)-4-acetamido-5-amino-3-(pentan-3-yloxy)cyclohex-1-ene-1-carboxylate, the structure is shown in formula 01:

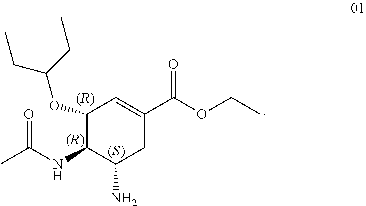

The chemical name of oseltamivir active metabolite is (3R,4R,5S)-4-acetamido-5-amino-3-(pentan-3-yloxy)cyclohex-1-ene-1-carboxylic acid, the structure is shown in formula 02:

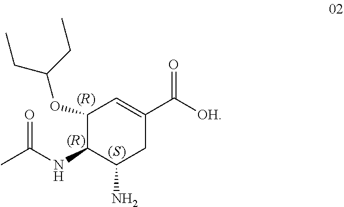

Oseltamivir phosphate is mainly used for the treatment of influenza A and B in adults and children aged 1 year and over, and used for the prevention of influenza A and B in adults and adolescents aged 13 years and over. The marketed dosage forms include capsules, dry suspensions, and granules. The strengths include 15 mg, 30 mg, 45 mg, 75 mg capsules or granules, and 6 mg/ml dry suspension based on oseltamivir, these dosage forms are conventional immediate-release formulation. The in vitro dissolution shows that they complete release within 10 minutes, rapid absorb after oral administration, and the taking method for the treatment of influenza is to take twice a day. The investigation and study found that the ability of oseltamivir phosphate to fight influenza is related to the maintenance time of effective plasma concentration in vivo. In order to maintain an effective plasma concentration, immediate-release formulation need to be taken several times a day, administered frequently, patient's compliance is poor, and it is easy to take untimely or often missed doses, which is resulting in poor efficacy. At the same time, for children's administration, the sustained-release formulation once a day can greatly increase the compliance of children's medication by reducing the times of medication. Therefore, the development of a sustained-release formulation of oseltamivir has great clinical significance.

From the prior art researches, it can be found that conventional sustained-release formulations usually have problems of time delay in absorption and reduced bioavailability. Oseltamivir phosphate is required to be taken within 48 hours after the onset of flu symptoms, and the sooner it is taken, the better. For sustained-release formulations with time delay in absorption, the optimal treatment time window may be missed, resulting in poor clinical efficacy. At the same time, the absorption rates of oseltamivir in the upper part of the small intestine, the lower part of the small intestine, and the colon are different. The absorption rate of the colon is significantly lower than that of the small intestine, and an inappropriate release rate will lead to a decrease in bioavailability and the long-term therapeutic effect cannot be achieved. However, in order to achieve a rapid onset of action, the sustained-release formulation usually results in a burst release, so that a long-term therapeutic effect cannot be achieved. Therefore, there is an urgent need to develop a sustained-release formulation that can take effect in a short time and maintain a long-term effect, but these formulations require a special release rate to achieve the expected clinical effect, and this special release rate is not obviously, it needs a lot of experimental explorations.

The purpose of the present invention is to develop an oseltamivir phosphate formulation that can take into account both the rapid onset of action and the maintenance of effective plasma concentration for a long time.

### SUMMARY

### Summary of the Invention

The first aspect of the present invention provides an oseltamivir formulation, which can be administered once a day and can achieve sustained release for at least 24 hours or longer, which can reduce the times of administration and avoid peak-to-valley fluctuations, thereby improving the compliance and safety of patients.

The second aspect of the present invention provides an oseltamivir formulation, which can be administered once a day. The formulation comprises a sustained-release part containing oseltamivir or salt thereof and an immediate-release part containing oseltamivir or salt thereof. In the formulation, the ratio of the immediate-release part and the sustained-release part will significantly affect the onset time and the maintenance time of the steady-state plasma concentration, as well as the safety of medication. When the ratio of immediate-release part and sustained-release part is low, the effective plasma concentration cannot be reached quickly, resulting onset slowly. While when the ratio of immediate-release part and sustained-release part is high, although it can quickly reach the effective plasma concentration, it may also lead to excessive concentration, increasing the probability of adverse reactions (gastrointestinal mucosal irritation). In addition, when the ratio of immediate-release part and sustained-release part is low, the effective plasma concentration can only be maintained for a short time, resulting in the medication frequency of once-a-day cannot be maintained throughout the day, so that the sustained release effect cannot be achieved. Therefore, the ratio of the immediate-release part and sustained-release part is very critical to the clinical effect.

The oseltamivir formulation provided herein is administered once a day, can be a biphasic release formulation, a three-phase release formulation, or a multiphase release formulation with more than three phases.

The third aspect of the present invention provides a preparation method of the oseltamivir formulation. The oseltamivir formulation prepared by the method has good in vitro and in vivo effect data, stable quality and meets quality requirements; the method is relatively simple to operate, and has good reproducibility, and is suitable for large-scale industrial manufacture.

### Definition of Terms

The invention is intended to cover all alternatives, modifications, and equivalents which may be included within the scope of the present invention as defined by the claims. One skilled in the art will recognize many methods and materials similar or equivalent to those described herein, which could be used in the practice of the present invention. The present invention is in no way limited to the methods and materials described herein. In the event that one or more of the incorporated literature, patents, and similar materials differs from or contradicts this application, including but not limited to defined terms, term usage, described techniques, or the like, this application controls.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

Unless defined otherwise, all technical and scientific terms used herein have the same meaning as is commonly understood by one skilled in the art to which this invention belongs. All patents and publications referred to herein are incorporated by reference in their entirety.

The term “comprise” or “include” or “contain” is an open expression, it means comprising the contents disclosed herein, but don't exclude other contents.

In the present invention, regardless of whether the words “about” or “approximately” are used, all numbers disclosed herein are approximate values. The value of each number may vary by less than 10%, or a reasonable difference that one skilled in the art would consider, such as 1%, 2%, 3%, 4% or 5%.

The term “Cmax” refers to the maximum plasma concentration in vivo after administration.

The term “C2h” refers to the corresponding plasma concentration 2 hours after administration.

The term “Tmax” refers to the time corresponding to the maximum plasma concentration in vivo after administration.

The term “AUC₀₋ₜ” refers to the area under the plasma concentration-time curve from 0 to the last selected time point after administration.

The term “BA” refers to bioavailability.

Concentration “ng/mL” refers to nanograms/mL, which is weight/volume, the volume is plasma volume.

“ng·h/mL” refers to nanograms per hour/mL, which is weight time/volume.

“Oseltamivir active metabolite” refers to is (3R,4R,5S)-4-acetamido-5-amino-3-(pentan-3-yloxy)cyclohex-1-ene-1-carboxylic acid, the structure is shown in formula 02.

“Sustained-release” refers to the plasma concentration of the oseltamivir active metabolite ((3R,4R,5S)-4-acetamido-5-amino-3-(pentan-3-yloxy)cyclohex-1-ene-1-carboxylic acid) of the sample detected by LC/MS/MS analytical instrument according to its detection limit.

LC/MS/MS refers to liquid chromatography-mass spectrometry.

“HPLC” refers to High Performance Liquid Chromatography.

“Peak-to-valley ratio of plasma concentration” refers to the ratio of the highest plasma concentration in vivo within 24 hours after administration to the plasma concentration at 24 hours.

ng refers to nanograms, mg refers to milligrams, h refers to hours, mm refers to millimeters, ° C. refers to degrees Celsius, and rpm refers to revolutions per minute.

Kollicoat® SR 30D refers to a 30% dispersion of polyvinyl acetate, Kollidon® SR refers to a blend of polyvinyl acetate and povidone.

### DETAILED DESCRIPTION OF THE INVENTION

Based on the deficiencies of the prior art, the present invention prepares a formulation containing oseltamivir or salt thereof after in-depth research and investigation. The formulation is administered once a day, and a sustained release of at least 24 hours or longer can be obtained, which can reduce the times of administration and avoid peak-to-valley fluctuations, thereby improving the treatment compliance and safety of patients. The formulation can be a single-phase release formulation, a biphasic release formulation, a three-phase release formulation, or a multi-phase release formulation with more than three phases. The formulation can maintain a relatively high plasma concentration in vivo for a long time, and has high bioavailability.

In the first aspect of the present invention, provided herein is an oseltamivir formulation, comprising oseltamivir or salt thereof, and the formulation is administered once a day. In some embodiments, oseltamivir is included; in some embodiments, oseltamivir phosphate is included.

In the oseltamivir formulation, the active ingredient oseltamivir or salt thereof can be rapidly released within a certain time period of ≤4 h. In some embodiments, in the oseltamivir formulation, the active ingredient oseltamivir or salt thereof can be rapidly released within a certain 1 h time period of ≤4 h. In some embodiments, in the oseltamivir formulation, the active ingredient oseltamivir or salt thereof is rapidly released within a certain 1 h time period of ≤4 h, and the release amount is 25%-55%; in some embodiments, the release amount is 25%-35%; in some embodiments, the release amount is 25%-40%; in some embodiments, the release amount is 25%-45%; in some embodiments, the release amount is 35%-40%; in some embodiments, the release amount is 35%-45%; in some embodiments, the release amount is 35%-55%; in some embodiments, the release amount is 40%-45%; in some embodiments, the release amount is 40%-55%; in some embodiments, the release amount is 45%-55%. In some embodiments, the release amount of the active ingredient oseltamivir or salt thereof within a certain 1 h time period of ≤4 h is 25%, 35%, 40%, 45% or 55%.

In some embodiments, in the oseltamivir formulation, the active ingredient oseltamivir or salt thereof is rapidly released within the first hour, and the release amount is 25%-55%. In some embodiments, the release amount is 25%-35%; in some embodiments, the release amount is 25%-40%; in some embodiments, the release amount is 25%-45%; in some embodiments, the release amount is 35%-40%; in some embodiments, the release amount is 35%-45%; in some embodiments, the release amount is 35%-55%; in some embodiments, the release amount is 40%-45%; in some embodiments, the release amount is 40%-55%; in some embodiments, the release amount is 45%-55%. In some embodiments, the release amount of the active ingredient oseltamivir or salt thereof at the first hour is 25%, 35%, 40%, 45%, or 55%.

In some embodiments, in the oseltamivir formulation, the release amount of active ingredient oseltamivir or salt thereof at 4 h is 25%-90%, or 25%-85%. In some embodiments, the release amount is 25%-53%; in some embodiments, the release amount is 25%-62%; in some embodiments, the release amount is 25%-78%; in some embodiments, the release amount is 53%-62%; in some embodiments, the release amount is 53%-78%; in some embodiments, the release amount is 53%-90%; in some embodiments, the release amount is 62%-78%; in some embodiments, the release amount is 62%-90%; in some embodiments, the release amount is 78%-90%. In some embodiments, the release amount of active ingredient oseltamivir or salt thereof at 4 h is 25%, 53%, 62%, 63%, 78% or 90%.

In some embodiments, in the oseltamivir formulation, the release amount of active ingredient oseltamivir or salt thereof at 10 h is greater than 70%. In some embodiments, in the oseltamivir formulation, the release amount of active ingredient oseltamivir or salt thereof at 10 h is 70%-99%. In some embodiments, in the oseltamivir formulation, the release amount of active ingredient oseltamivir or salt thereof at 10 h is greater than 74%, or greater than 77%, or greater than 80%, or greater than 86%, or greater than 89%, or greater than 93%, or greater than 95%, or greater than 98%. In some embodiments, in the oseltamivir formulation, the release amount of active ingredient oseltamivir or salt thereof at 10 h is 93%, 94%, 95%, 96%, 97%, 98%, 77%, 86%, 74% or 89%.

The oseltamivir formulation, the weight ratio of the active ingredient oseltamivir is 3%-50% of the total weight of the formulation. In some embodiments, in the oseltamivir formulation, the weight ratio of the active ingredient oseltamivir is 3%-45%. In some embodiments, the weight ratio of oseltamivir is 3%-21%; in some embodiments, the weight ratio of oseltamivir is 3%-32%; in some embodiments, the weight ratio of oseltamivir is 21%-32%; in some embodiments, the weight ratio of oseltamivir is 21%-50%; in some embodiments, the weight ratio of oseltamivir or salt thereof is 32%-50%. In some embodiments, the weight ratio of oseltamivir is 3%, 21%, 32% or 50%.

The oseltamivir formulation, after administration once a day, the peak-to-valley ratio of the plasma concentration in vivo of the oseltamivir active metabolite within 24 h is less than 2.5:1. The peak-to-valley ratio of plasma concentration in vivo is less than 2.5:1, which can avoid peak-to-valley fluctuations, thereby improving the treatment compliance and safety of patients. In some embodiments, the peak-to-valley ratio of the plasma concentration in vivo of the oseltamivir active metabolite within 24 h is 2.14:1. In some embodiments, the oseltamivir formulation, after administration once a day, the peak-to-valley ratio of the plasma concentration in vivo of the oseltamivir active metabolite within 24 h is less than 2:1, which is more conducive to avoiding peak-to-valley fluctuations, thereby improving the treatment compliance and safety of patients. In some embodiments, the peak-to-valley ratio of the plasma concentration in vivo of the oseltamivir active metabolite within 24 h is 1.21:1.

According to the weight of oseltamivir, the oseltamivir formulation has a single-dose strength of 60 mg-300 mg. In some embodiments, the strength is 60 mg-90 mg; in some embodiments, the strength is 60 mg-150 mg; in some embodiments, the strength is 60 mg-200 mg; in some embodiments, the strength is 90 mg-150 mg; in some embodiments, the strength is 90 mg-200 mg; in some embodiments, the strength is 90 mg-300 mg; in some embodiments, the strength is 150 mg-200 mg; in some embodiments, the strength is 150 mg-300 mg; in some embodiments, the strength is 200 mg-300 mg. In some embodiments, the single-dose strength is 60 mg, 90 mg, 150 mg, 200 mg or 300 mg. The current marketed immediate-release dosage form for children, according to the weight of oseltamivir, has a minimum strength of 30 mg, administered twice a day. If it is prepared as a sustained-release formulation administered once a day, the minimum strength is 60 mg. The currently marketed adult immediate-release dosage form, according to the weight of oseltamivir, the minimum strength is 75 mg, administered twice a day. If it is prepared as a sustained-release formulation administered once a day, the minimum strength is 150 mg. If the strength is increased to 300 mg, the effect cannot be equivalent with the immediate-release dosage form of 75 mg strength and administered twice a day, which indicates that the bioavailability is low, if there is a burst release, the risk of toxic and side effects will increase, and the tablet is heavy, causing difficulty in swallowing. Therefore, according to the weight of oseltamivir, 60 mg-300 mg is a more appropriate strength.

The oseltamivir formulation further comprises at least one sustained-release material, and the weight ratio of the sustained-release material is 3%-50% of the total weight of the formulation. The weight ratio of the sustained-release material is 3%-50%, and a formulation that can continuously release oseltamivir for at least 24 h or longer can be prepared, and the times of administration can be reduced. In some embodiments, the weight ratio of the sustained-release material is 3%-10%; in some embodiments, the weight ratio of the sustained-release material is 3%-20%; in some embodiments, the weight ratio of the sustained-release material is 3%-30%; in some embodiments, the weight ratio of the sustained-release material is 10%-20%; in some embodiments, the weight ratio of the sustained-release material is 10%-30%; in some embodiments, the weight ratio of the sustained-release material is 10%-50%; in some embodiments, the weight ratio of the sustained-release material is 20%-30%; in some embodiments, the weight ratio of the sustained-release material is 20%-50%. In some embodiments, the weight ratio of the sustained-release material is 30%-50%. In some embodiments, the weight ratio of the sustained-release material is 3.3%, 10%, 20%, 29% or 50%.

In some embodiments, the oseltamivir formulation further comprises at least one sustained-release material, the sustained-release material includes at least one selected from ethyl cellulose, hydroxypropyl methyl cellulose, cellulose acetate, poly oxyethylene, polyvinyl alcohol, methacrylic acid-ethyl acrylate copolymer RL, methacrylic acid-ethyl acrylate copolymer RS, methacrylic acid-ethyl acrylate copolymer NE 30D, methacrylic acid-ethyl acrylate copolymer NE L100-55, hypromellose acetate succinate, glyceryl behenate, chitosan, carbomer, carnauba wax, Kollicoat® SR 30D, Kollidon® SR, cellulose acetate titanate, sodium alginate, sodium carboxymethyl cellulose, carrageenan, cetyl alcohol, stearyl alcohol, cetostearyl alcohol, paraffin. In some embodiments, the sustained-release material is cellulose acetate; in some embodiments, the sustained-release material is methacrylic acid-ethyl acrylate copolymer RL and methacrylic acid-ethyl acrylate copolymer RS; in some embodiments, the sustained-release material is ethyl cellulose; in some embodiments, the sustained-release material is hydroxypropyl methylcellulose; in some embodiments, the sustained-release material is poly oxyethylene and ethyl cellulose.

In some embodiments, the oseltamivir formulation further comprises at least one sustained-release material, and the sustained-release material includes a pH-independent polymer material, a pH-dependent polymer material or a waxy matrix material. The pH-independent polymer material includes at least one selected from ethyl cellulose, hydroxypropyl methyl cellulose, cellulose acetate, poly oxyethylene, polyvinyl alcohol, methacrylic acid-ethyl acrylate copolymer RL, methacrylic acid-ethyl acrylate copolymer RS, methacrylic acid-ethyl acrylate copolymer NE 30D, Kollicoat® SR 30D, Kollidon® SR; the pH-dependent polymer material includes at least one selected from Methacrylic acid-ethyl acrylate copolymer L100-55, hypromellose acetate succinate, cellulose acetate titanate, chitosan, carbomer, carrageenan, sodium alginate, sodium carboxymethyl cellulose; the waxy matrix material includes at least one selected from glyceryl behenate, carnauba wax, cetyl alcohol, stearyl alcohol, cetostearyl alcohol, and paraffin.

In some embodiments, the oseltamivir formulation comprises at least one sustained-release material, and the weight ratio of oseltamivir or salt thereof is 3%-50% of the total weight of the formulation. In some embodiments, the oseltamivir formulation comprises at least one sustained-release material, and the release amount of oseltamivir or salt thereof at 4 h is 25%-90%. In some embodiments, the oseltamivir formulation comprises at least one sustained-release material, and the release amount of oseltamivir or salt thereof at 10 h is greater than 70%. In some embodiments, the oseltamivir formulation comprises at least one sustained-release material, the weight ratio of oseltamivir or salt thereof is 3%-50%, and the release amount of oseltamivir or salt thereof at 4 h is 25%-90%. In some embodiments, the oseltamivir formulation comprises at least one sustained-release material, the weight ratio of oseltamivir or salt thereof is 3%-50%, and the release amount of oseltamivir or salt thereof at 10 h is greater than 70%. In some embodiments, in the oseltamivir formulation, the release amount of oseltamivir or salt thereof at 4 h is 25%-90%, and the release amount of oseltamivir or salt thereof at 10 h is greater than 70%. In some embodiments, the oseltamivir formulation comprises at least one sustained-release material, the release amount of oseltamivir or salt thereof at 4 h is 25%-90%, and the release amount of oseltamivir or salt thereof at 10 h is greater than 70%. In some embodiments, the oseltamivir formulation comprises at least one sustained-release material; the weight ratio of oseltamivir or salt thereof is 3%-50%; the release amount of oseltamivir or salt thereof at 4 h is 25%-90%, and the release amount of oseltamivir or salt thereof at 10 h is greater than 70%.

In some embodiments, the sustained-release material in the oseltamivir formulation is cellulose acetate, and the weight ratio of oseltamivir or salt thereof is 25%-35% of the total weight of the formulation. In some embodiments, the sustained-release material in the oseltamivir formulation is cellulose acetate, and the weight ratio of oseltamivir or salt thereof is 30%-35%. In some embodiments, the sustained-release material in the oseltamivir formulation is cellulose acetate, and the weight ratio of oseltamivir or salt thereof is 31.6%. In some embodiments, the sustained-release material in the oseltamivir formulation is cellulose acetate, the release amount of oseltamivir or salt thereof at 4 h is 25%-90%. In some embodiments, the sustained-release material in the oseltamivir formulation is cellulose acetate, the release amount of oseltamivir or salt thereof at 4 h is greater than 70%. In some embodiments, the sustained-release material in the oseltamivir formulation is cellulose acetate, the release amount of oseltamivir or salt thereof at 10 h is greater than 70% or 90% or 95%. In some embodiments, the sustained-release material in the oseltamivir formulation is cellulose acetate, the release amount of oseltamivir or salt thereof at 4 h is greater than 70%, the release amount of oseltamivir or salt thereof at 10 h is greater than 70% or 90% or 95%. In some embodiments, the sustained-release material in the oseltamivir formulation is cellulose acetate, the weight ratio of oseltamivir or salt thereof is 30%-35%, the release amount of oseltamivir or salt thereof at 4 h is 25%-90%, the release amount of oseltamivir or salt thereof at 10 h is greater than 70% or 80% or 90% or 95%. In some embodiments, the sustained-release material in the oseltamivir formulation is cellulose acetate, the weight ratio of oseltamivir or salt thereof is 30%-35%, the release amount of oseltamivir or salt thereof at 4 h is greater than 70%, the release amount of oseltamivir or salt thereof at 10 h is greater than 70% or 80% or 90% or 95%. In some embodiments, the sustained-release material in the oseltamivir formulation is cellulose acetate, the weight ratio of oseltamivir or salt thereof is 31.6%, the release amount of oseltamivir at 4 h is 78%, the release amount of oseltamivir or salt thereof at 10 h is 97%. In some embodiments, the sustained-release material in the oseltamivir formulation is hydroxypropyl methyl cellulose, the weight ratio of oseltamivir or salt thereof is 32.0%, the release amount of oseltamivir at 4 h is 63%, the release amount of oseltamivir or salt thereof at 10 h is 86%.

In some embodiments, the oseltamivir formulation further comprises at least one sustained-release material, the weight ratio of the sustained-release material is 3%-50% of the total weight of the formulation, and the sustained-release material includes at least one selected from ethyl cellulose, hydroxypropyl methyl cellulose, cellulose acetate, poly oxyethylene, polyvinyl alcohol, methacrylic acid-ethyl acrylate copolymer RL, methacrylic acid-ethyl acrylate copolymer RS, methacrylic acid-ethyl acrylate copolymer NE 30 D, methacrylic acid-ethyl acrylate copolymer L100-55, hypromellose acetate succinate, glyceryl behenate, chitosan, carbomer, carnauba wax, Kollicoat® SR 30D, Kollidon® SR, cellulose acetate titanate, sodium alginate, sodium carboxymethyl cellulose, carrageenan, cetyl alcohol, stearyl alcohol, cetostearyl alcohol, paraffin.

In some embodiments, in the oseltamivir formulation, the release amount of oseltamivir or salt thereof at 1 h is 25%-55%, the release amount of oseltamivir or salt thereof at 4 h is 25%-90%, the release amount of oseltamivir or salt thereof at 10 h is greater than 70%; the weight ratio of oseltamivir or salt thereof is 3%-50%; according to the weight of oseltamivir, the strength is 60 mg-300 mg; after administration once a day, the peak-to-valley ratio of the in vivo plasma concentration of the oseltamivir active metabolite within 24 h is less than 2.5:1; optionally, in some embodiments, the oseltamivir formulation further comprises at least one sustained-release material, and the weight ratio of the sustained-release material is 3%-50%, and the sustained-release material includes at least one selected from ethyl cellulose, hydroxypropyl methyl cellulose, cellulose acetate, poly oxyethylene, polyvinyl alcohol, methacrylic acid-ethyl acrylate copolymer RL, methacrylic acid-ethyl acrylate copolymer RS, methacrylic acid-ethyl acrylate copolymer NE 30D, methacrylic acid-ethyl acrylate copolymer NE L100-55, hypromellose acetate succinate, glyceryl behenate, chitosan, carbomer, carnauba wax, Kollicoat® SR 30D, Kollidon® SR, cellulose acetate titanate, sodium alginate, sodium carboxymethyl cellulose, carrageenan, cetyl alcohol, stearyl alcohol, cetostearyl alcohol, paraffin.

In the second aspect of the present invention, provided herein is an oseltamivir formulation, comprising oseltamivir or salt thereof, the formulation is administered once a day, and the formulation comprises a sustained-release part containing oseltamivir or salt thereof and an immediate-release part containing oseltamivir or salt thereof. The formulation can be a biphasic release formulation, a three-phase release formulation, or a multiphase release formulation with more than three phases. In some embodiments, the formulation is a biphasic release formulation, in some embodiments, the formulation is a three-phase release formulation, and in some embodiments, the formulation is a multiphase release formulation. In some embodiments, the active ingredient is oseltamivir; in some embodiments, the active ingredient is oseltamivir phosphate.

In the oseltamivir formulation comprising the sustained-release part containing oseltamivir or salt thereof and the immediate-release part containing oseltamivir or salt thereof, the active ingredient oseltamivir or salt thereof in a time period of ≤4 h can be quickly released, and the release amount is 25%-55%. In the oseltamivir formulation comprising the sustained-release part containing oseltamivir or salt thereof and the immediate-release part containing oseltamivir or salt thereof, the active ingredient oseltamivir or salt thereof can be rapidly released within 1 h time period of ≤4 h, the release amount is 25%-55%; in some embodiments, the release amount is 25%-35%; in some embodiments, the release amount is 25%-40%; in some embodiments, the release amount is 25%-45%; in some embodiments, the release amount is 35%-40%; in some embodiments, the release amount is 35%-45%; in some embodiments, the release amount is 35%-55%; in some embodiments, the release amount is 40%-45%; in some embodiments, the release amount is 40%-55%; in some embodiments, the release amount is 45%-55%. In some embodiments, the release amount of the active ingredient oseltamivir or salt thereof within a certain 1 h time period of ≤4 h is 25%, 35%, 40%, 45% or 55%.

In some embodiments, in the oseltamivir formulation, the active ingredient oseltamivir or salt thereof is rapidly released in the first hour, and the release amount is 25%-55%; in some embodiments, the release amount is 25%-35%; in some embodiments, the release amount is 25%-40%; in some embodiments, the release amount is 25%-45%; in some embodiments, the release amount is 35%-40%; in some embodiments, the release amount is 35%-45%; in some embodiments, the release amount is 35%-55%; in some embodiments, the release amount is 40%-45%; in some embodiments, the release amount is 40%-55%; in some embodiments, the release amount is 45%-55%. In some embodiments, the release amount of the active ingredient oseltamivir or salt thereof at 1 h is 25%, 35%, 40%, 45%, or 55%.

In some embodiments, in the oseltamivir formulation, the release amount of the active ingredient oseltamivir or salt thereof at 4 h is 25%-90%, or 25%-85%; in some embodiments, the release amount is 25%-53%; in some embodiments, the release amount is 25%-62%; in some embodiments, the release amount is 25%-78%; in some embodiments, the release amount is 53%-62%; in some embodiments, the release amount is 53%-78%; in some embodiments, the release amount is 53%-90%; in some embodiments, the release amount is 62%-78%; in some embodiments, the release amount is 62%-90%; in some embodiments, the release amount is 78%-90%. In some embodiments, the release amount of active ingredient oseltamivir or salt thereof at 4 h is 25%, 53%, 62%, 63%, 78% or 90%.

In some embodiments, in the oseltamivir formulation, the release amount of active ingredient oseltamivir or salt thereof at 10 h is greater than 70%. In some embodiments, in the oseltamivir formulation, the release amount of active ingredient oseltamivir or salt thereof at 10 h is 70%-99%. In some embodiments, in the oseltamivir formulation, the release amount of active ingredient oseltamivir or salt thereof at 10 h is greater than 74%, or greater than 77%, or greater than 80%, or greater than 86%, or greater than 89%, or greater than 93%, or greater than 95%, or greater than 98%. In some embodiments, in the oseltamivir formulation, the release amount of active ingredient oseltamivir or salt thereof at 10 h is 93%, 94%, 95%, 96%, 97%, 98%, 77%, 86%, 74% or 89%.

The oseltamivir formulation, the weight ratio of the active ingredient oseltamivir is 3%-50%, or 3%-45% of the total weight of the formulation. In some embodiments, the weight ratio of oseltamivir is 3%-21%; in some embodiments, the weight ratio of oseltamivir is 3%-32%; in some embodiments, the weight ratio of oseltamivir is 21%-32%; in some embodiments, the weight ratio of oseltamivir is 21%-50%; in some embodiments, the weight ratio of oseltamivir or salt thereof is 32%-50%. In some embodiments, the weight ratio of oseltamivir is 3%, 21%, 32% or 50%.

The oseltamivir formulation, after administration once a day, the peak-to-valley ratio of the plasma concentration in vivo of the oseltamivir active metabolite within 24 h is less than 2.5:1. The peak-to-valley ratio of plasma concentration in vivo is less than 2.5:1, which can avoid peak-to-valley fluctuations, thereby improving the treatment compliance and safety of patients. In some embodiments, the peak-to-valley ratio of the plasma concentration in vivo of the oseltamivir active metabolite within 24 h is 2.14:1. In some embodiments, the oseltamivir formulation, after administration once a day, the peak-to-valley ratio of the plasma concentration in vivo of the oseltamivir active metabolite within 24 h is less than 2:1, which is more conducive to avoiding peak-to-valley fluctuations, thereby improving the treatment compliance and safety of patients. In some embodiments, the peak-to-valley ratio of the plasma concentration in vivo of the oseltamivir active metabolite within 24 h is 1.21:1.

According to the weight of oseltamivir, the single-dose strength of the oseltamivir formulation is 60 mg-300 mg; in some embodiments, the strength is 60 mg-90 mg; in some embodiments, the strength is 60 mg-150 mg; in some embodiments, the strength is 60 mg-200 mg; in some embodiments, the strength is 90 mg-150 mg; in some embodiments, the strength is 90 mg-200 mg; in some embodiments, the strength is 90 mg-300 mg; in some embodiments, the strength is 150 mg-200 mg; in some embodiments, the strength is 150 mg-300 mg; in some embodiments, the strength is 200 mg-300 mg. In some embodiments, the strength is 60 mg, 90 mg, 150 mg, 200 mg or 300 mg. The current marketed immediate-release dosage form for children, according to the weight of oseltamivir, has a minimum strength of 30 mg, administered twice a day. If it is prepared as a sustained-release formulation administered once a day, the minimum strength is 60 mg. The currently marketed adult immediate-release dosage form, according to the weight of oseltamivir, the minimum strength is 75 mg, administered twice a day. If it is prepared as a sustained-release formulation administered once a day, the minimum strength is 150 mg, if the strength is increased to 300 mg, the effect cannot be equivalent with the immediate-release dosage form of 75 mg strength and administered twice a day, which indicates that the bioavailability is low, if there is a burst release, the risk of toxic and side effects will increase, and the tablet is heavy, causing difficulty in swallowing. Therefore, according to the weight of oseltamivir, 60 mg-300 mg is a more appropriate strength.

The oseltamivir formulation further comprises at least one sustained-release material, and the weight ratio of the sustained-release material is 3%-50% of the total weight of the formulation. In some embodiments, the weight ratio of the sustained-release material is 3%-10%; in some embodiments, the weight ratio of the sustained-release material is 3%-20%; in some embodiments, the weight ratio of the sustained-release material is 3%-30%; in some embodiments, the weight ratio of the sustained-release material is 10%-20%; in some embodiments, the weight ratio of the sustained-release material is 10-30%; in some embodiments, the weight ratio of the sustained-release material is 10%-50%; in some embodiments, the weight ratio of the sustained-release material is 20%-30%; in some embodiments, the weight ratio of the sustained-release material is 20%-50%; in some embodiments, the weight ratio of the sustained-release material is 30%-50%. In some embodiments, the weight ratio of the sustained-release material is 3.3%, 10%, 20%, 29% or 50%. The weight ratio of the sustained-release material is 3%-50%, and a formulation that can continuously release oseltamivir for at least 24 h or longer can be prepared, and the times of administration can be reduced.

In some embodiments, the oseltamivir formulation further comprises at least one sustained-release material, the sustained-release material includes at least one selected from ethyl cellulose, hydroxypropyl methyl cellulose, cellulose acetate, polyoxy ethylene, polyvinyl alcohol, methacrylic acid-ethyl acrylate copolymer RL, methacrylic acid-ethyl acrylate copolymer RS, methacrylic acid-ethyl acrylate copolymer NE 30D, methacrylic acid-ethyl acrylate copolymer L100-55, hypromellose acetate succinate, glyceryl behenate, chitosan, carbomer, carnauba wax, Kollicoat® SR 30D, Kollidon® SR, cellulose acetate titanate, sodium alginate, sodium carboxymethyl cellulose, carrageenan, cetyl alcohol, stearyl alcohol, cetostearyl alcohol and paraffin. In some embodiments, the sustained-release material is cellulose acetate; in some embodiments, the sustained-release material is methacrylic acid-ethyl acrylate copolymer RL and methacrylic acid-ethyl acrylate copolymer RS; in some embodiments, the sustained-release material is ethyl cellulose; in some embodiments, the sustained-release material is hydroxypropyl methylcellulose; in some embodiments, the sustained-release material is poly oxyethylene and ethyl cellulose.

In some embodiments, the oseltamivir formulation further comprises at least one sustained-release material, and the sustained-release material includes a pH-independent polymer material, a pH-dependent polymer material or a waxy matrix material. The pH-independent polymer material includes at least one selected from ethyl cellulose, hydroxypropyl methyl cellulose, cellulose acetate, poly oxyethylene, polyvinyl alcohol, methacrylic acid-ethyl acrylate copolymer RL, methacrylic acid-ethyl acrylate copolymer RS, methacrylic acid-ethyl acrylate copolymer NE 30D, Kollicoat® SR 30D, Kollidon® SR; the pH-dependent polymer material includes at least one selected from methacrylic acid-ethyl acrylate copolymer L100-55, hypromellose acetate succinate, cellulose acetate titanate, chitosan, carbomer, carrageenan, sodium alginate, sodium carboxymethyl cellulose; the waxy matrix material includes at least one selected from glyceryl behenate, carnauba wax, cetyl alcohol, stearyl alcohol, cetostearyl alcohol and paraffin.

In some embodiments, the oseltamivir formulation comprises at least one sustained-release material, and the weight ratio of oseltamivir is 3%-50% of the total weight of the formulation; the release amount of oseltamivir or salt thereof at 4 h is 25%-90%, and the release amount of oseltamivir or salt thereof at 10 h is greater than 70%. In some embodiments, the sustained-release materials in the oseltamivir formulation are methacrylic acid-ethyl acrylate copolymer RL and methacrylic acid-ethyl acrylate copolymer RS, and the weight ratio of oseltamivir is 22.4%, the release amount of oseltamivir at 4 h is 78%, the release amount of oseltamivir or salt thereof at 10 h is 97%; in some embodiments, the sustained-release material in the oseltamivir formulation is ethyl cellulose, and the weight ratio of oseltamivir is 3.3%, the release amount of oseltamivir at 4 h is 75%, and the release amount of oseltamivir or salt thereof at 10 h is 96%.

In some embodiments, the oseltamivir formulation further comprises at least one sustained-release material, and the weight ratio of the sustained-release material is 3%-50% of the total weight of the formulation, and the sustained-release material includes at least one selected from ethyl cellulose, hydroxypropyl methyl cellulose, cellulose acetate, polyoxyethylene, polyvinyl alcohol, methacrylic acid-ethyl acrylate copolymer RL, methacrylic acid-ethyl acrylate copolymer RS, methacrylic acid-ethyl acrylate copolymer NE 30D, methacrylic acid-ethyl acrylate copolymer L100-55, hypromellose acetate succinate, glyceryl behenate, chitosan, carbomer, carnauba wax, Kollicoat® SR 30D, Kollidon® SR, cellulose acetate titanate, sodium alginate, sodium carboxymethyl cellulose, carrageenan, cetyl alcohol, stearyl alcohol, cetostearyl alcohol and paraffin.

In some embodiments, in the oseltamivir formulation, the release amount of oseltamivir or salt thereof at 1 h is 25%-55%, the release amount of oseltamivir or salt thereof at 4 h is 25%-90%, the release amount of oseltamivir or salt thereof at 10 h is greater than 70%; the weight ratio of oseltamivir or salt thereof is 3%-50%; according to the weight of oseltamivir, the single-dose strength is 60 mg-300 mg; after administration once a day, the peak-to-valley ratio of the plasma concentration in vivo of the oseltamivir active metabolite within 24 h is less than 2.5:1. Optionally, in some embodiments, the oseltamivir formulation further comprises at least one sustained-release material, and the weight ratio of the sustained-release material is 3%-50%, and the sustained-release material includes at least one selected from ethyl cellulose, hydroxypropyl methyl cellulose, cellulose acetate, polyoxyethylene, polyvinyl alcohol, methacrylic acid-ethyl acrylate copolymer RL, methacrylic acid-ethyl acrylate copolymer RS, methacrylic acid-ethyl acrylate copolymer NE 30D, methacrylic acid-ethyl acrylate copolymer L100-55, hypromellose acetate succinate, glyceryl behenate, chitosan, carbomer, carnauba wax, Kollicoat® SR 30D, Kollidon® SR, cellulose acetate titanate, sodium alginate, sodium carboxymethyl cellulose, carrageenan, cetyl alcohol, stearyl alcohol, cetostearyl alcohol and paraffin.

The oseltamivir formulation, the oseltamivir strength in the immediate-release part is 10 mg-75 mg, or 15 mg-75 mg, or 15 mg-30 mg, or 15 mg-50 mg, or 30 mg-50 mg, or 30 mg-75 mg, or 50 mg-75 mg; the oseltamivir strength in the sustained-release part is 30 mg-225 mg, or 30 mg-175 mg, or 50 mg-100 mg, or 50 mg-120 mg, or 50 mg-175 mg, or 50 mg-225 mg, or 100 mg-120 mg, or 100 mg-150 mg, or 100 mg-225 mg, or 120 mg-150 mg, or 120 mg-175 mg, or 120 mg-225 mg, or 150 mg-175 mg, or 150 mg-225 mg, or 175 mg-225 mg. In some embodiments, the oseltamivir strength in the immediate-release part is 50 mg, the oseltamivir strength in the sustained-release part is 100 mg; in some embodiments, the oseltamivir strength in the immediate-release part is 50 mg, the oseltamivir strength in the sustained-release part is 130 mg; in some embodiments, the oseltamivir strength in the immediate-release part is 30 mg, the oseltamivir strength in the sustained-release part is 60 mg; in some embodiments, the oseltamivir strength in the immediate-release part is 50 mg, the oseltamivir strength in the sustained-release part is 150 mg; in some embodiments, the oseltamivir strength in the immediate-release part is 10 mg, the oseltamivir strength in the sustained-release part is 50 mg; in some embodiments, the oseltamivir strength in the immediate-release part is 75 mg, the oseltamivir strength in the sustained-release part is 225 mg; in some embodiments, the oseltamivir strength in the immediate-release part is 30 mg, the oseltamivir strength in the sustained-release part is 120 mg.

The oseltamivir formulation provided in the first or second aspect of the present invention is used for the treatment of adult influenza A or adult influenza B. After administration once a day, the plasma concentration in vivo of the oseltamivir active metabolite within 24 h is greater than 100 ng/ml, or greater than 150 ng/ml, or greater than 170 ng/ml, or greater than 200 ng/ml, or greater than 250 ng/ml. In some embodiments, the plasma concentration in vivo of the oseltamivir active metabolite within 24 h is greater than 172 ng/ml; in some embodiments, the plasma concentration in vivo of the oseltamivir active metabolite within 24 h is greater than 261 ng/ml.

The oseltamivir formulation provided in the first or second aspect of the present invention is used for the treatment of children influenza A or children influenza B. After administration once a day, the plasma concentration in vivo of the oseltamivir active metabolite within 24 h is greater than 100 ng/ml, or greater than 150 ng/ml, or greater than 200 ng/ml, or greater than 250 ng/ml.

The oseltamivir formulation provided in the first or second aspect of the present invention is used for the treatment of influenza A or influenza B in adults or children, and the strength is 60 mg-300 mg according to the weight of oseltamivir. In some embodiments, the strength of the formulation is 60 mg; in some embodiments, the strength of the formulation is 90 mg; in some embodiments, the strength of the formulation is 150 mg; in some embodiments, the strength of the formulation is 200 mg; in some embodiments, the strength of the formulation is 300 mg.

In some embodiments, the strength of the oseltamivir formulation is 150 mg-300 mg according to the weight of oseltamivir. In some embodiments, the oseltamivir formulation used for the treatment of adult influenza A or adult influenza B has a strength of 150 mg-300 mg according to the weight of oseltamivir. The currently marketed adult immediate-release dosage form has a minimum strength of 75 mg according to the weight of oseltamivir, and administered twice a day. If it is prepared as a sustained-release formulation administered once a day, the minimum strength is 150 mg, if the strength is increased to 300 mg, the effect cannot be equivalent with the immediate-release dosage form of 75 mg strength and administered twice a day, which indicates that the bioavailability is low, if there is a burst release, the risk of toxic and side effects will increase, and the tablet is heavy, causing difficulty in swallowing. Therefore, according to the weight of oseltamivir, 150 mg-300 mg is a more appropriate strength.

In some embodiments, the strength of the oseltamivir formulation is 60 mg-180 mg according to the weight of oseltamivir. In some embodiments, the oseltamivir formulation used for the treatment of influenza A or influenza B in children has a strength of 60 mg-180 mg according to the weight of oseltamivir. The strengths of the current marketed immediate-release dosage forms for children are 30 mg and 45 mg, administered twice a day and 30 mg or 45 mg each time. If it is prepared as a sustained-release formulation administered once a day, the ideal strength is 60-90 mg, but the bioavailability of the sustained-release formulation is generally lower than that of immediate-release formulation, it is necessary to appropriately increase the strength of sustained-release formulation to be bioequivalent to immediate-release formulation which is administered twice a day. However, the higher the dose, the higher the risk of general toxic and side effects. Based on the Roche's review report, the strength increased 2 times is within the acceptable range, therefore, 60-180 mg is a more appropriate strength for children's dosage form. The strength of the sustained-release formulation for children exceeds 180 mg, indicating that the bioavailability is low, if there is a burst release, the risk of toxic and side effects will increase.

For the oseltamivir formulation, after administration once a day, the plasma concentration in vivo of the oseltamivir active metabolite at 2 h is more than 20%, or more than 30%, or more than 40% of Cmax; the plasma concentration in vivo at 24 h is more than 30%, or more than 40% of Cmax. In some embodiments, the plasma concentration in vivo of the oseltamivir active metabolite at 2 h is 27% of Cmax; in some embodiments, the plasma concentration in vivo of the oseltamivir active metabolite at 2 h is 25% of Cmax; in some embodiments, the plasma concentration in vivo of the oseltamivir active metabolite at 24 h is 47% of Cmax; in some embodiments, the plasma concentration in vivo of the oseltamivir active metabolite at 24 h is 82% of Cmax.

The oseltamivir formulation provided by the first or second aspect of the present invention continuously releases oseltamivir for a period of at least 24 h.

For the oseltamivir formulation, the plasma concentration in vivo of the oseltamivir active metabolite is greater than 100 ng/ml, or greater than 150 ng/ml, and the maintenance time is greater than 16 h, or greater than 20 h. In some embodiments, for the oseltamivir formulation, the plasma concentration in vivo of the oseltamivir active metabolite is greater than or equal to 170 ng/ml, and the maintenance time is greater than 16 h. In some embodiments, for the oseltamivir formulation, the plasma concentration in vivo of the oseltamivir active metabolite is greater than or equal to 250 ng/ml, and the maintenance time is greater than 12 h. In some embodiments, for the oseltamivir formulation, the plasma concentration in vivo of the oseltamivir active metabolite is greater than or equal to 250 ng/ml, and the maintenance time is greater than 16 h. In some embodiments, the plasma concentration in vivo of the oseltamivir active metabolite is 261 ng/ml, and the maintenance time is 16 h; in some embodiments, the plasma concentration in vivo of the oseltamivir active metabolite is 172 ng/ml, and the maintenance time is 20 h.

The third aspect of the present invention provides an oseltamivir formulation, comprising oseltamivir or salt thereof, the formulation is administered once a day, and the formulation comprises a sustained-release part containing oseltamivir or salt thereof and an immediate-release part containing oseltamivir or salt thereof, according to the total weight of oseltamivir in the formulation, the weight ratio of oseltamivir in the immediate-release part is 20%-50%. In some embodiments, the weight ratio of oseltamivir in the immediate-release part is 20%-30%; in some embodiments, the weight ratio of oseltamivir in the immediate-release part is 20%-40%; in some embodiments, the weight ratio of oseltamivir in the immediate-release part is 30%-40%; in some embodiments, the weight ratio of oseltamivir in the immediate-release part is 30%-50%; in some embodiments, the weight ratio of oseltamivir in the immediate-release part is 40%-50%. The formulation can be a biphasic release formulation, a three-phase release formulation, or a multiphase release formulation with more than three phases. In some embodiments, the formulation is a biphasic release formulation, in some embodiments, the formulation is a three-phase release formulation, and in some embodiments, the formulation is a multiphase release formulation. In some embodiments, the active ingredient is oseltamivir; in some embodiments, the active ingredient is oseltamivir phosphate.

The fourth aspect of the present invention provides an oseltamivir formulation, comprising oseltamivir or salt thereof, the formulation is administered once a day, and the formulation comprises a sustained-release part containing oseltamivir or salt thereof and an immediate-release part containing oseltamivir or salt thereof, the weight ratio of oseltamivir in the immediate-release part and in the sustained-release part is 1:4-1:1. In some embodiments, the weight ratio of oseltamivir in the immediate-release part and in the sustained-release part is 1:4-1:3; in some embodiments, the weight ratio of oseltamivir in the immediate-release part and in the sustained-release part is 1:4-1:2; in some embodiments, the weight ratio of oseltamivir in the immediate-release part and in the sustained-release part is 1:3-1:2; in some embodiments, the weight ratio of oseltamivir in the immediate-release part and in the sustained-release part is 1:3-1:1; in some embodiments, the weight ratio of oseltamivir in the immediate-release part and in the sustained-release part is 1:2-1:1; in some embodiments, the weight ratio of oseltamivir in the immediate-release part and in the sustained-release part is 1:4, 1:3, 1:2 or 1:1. The formulation can be a biphasic release formulation, a three-phase release formulation, or a multiphase release formulation with more than three phases. In some embodiments, the formulation is a biphasic release formulation, in some embodiments, the formulation is a three-phase release formulation, and in some embodiments, the formulation is a multiphase release formulation. In some embodiments, the active ingredient is oseltamivir; in some embodiments, the active ingredient is oseltamivir phosphate.

The fifth aspect of the present invention provides an oseltamivir formulation, comprising oseltamivir or salt thereof, the formulation is administered once a day, and the formulation comprises a sustained-release part containing oseltamivir or salt thereof and an immediate-release part containing oseltamivir or salt thereof. According to the total weight of oseltamivir in the formulation, the weight ratio of oseltamivir in the immediate-release part is 20%-50%, the weight ratio of oseltamivir in the immediate-release part and in the sustained-release part is 1:4-1:1. In some embodiments, the weight ratio of oseltamivir in the immediate-release part is 20%-30%; in some embodiments, the weight ratio of oseltamivir in the immediate-release part is 20%-40%; in some embodiments, the weight ratio of oseltamivir in the immediate-release part is 30%-40%; in some embodiments, the weight ratio of oseltamivir in the immediate-release part is 30%-50%; in some embodiments, the weight ratio of oseltamivir in the immediate-release part is 40%-50%. In some embodiments, the weight ratio of oseltamivir in the immediate-release part and in the sustained-release part is 1:4-1:3; in some embodiments, the weight ratio of oseltamivir in the immediate-release part and in the sustained-release part is 1:4-1:2; in some embodiments, the weight ratio of oseltamivir in the immediate-release part and in the sustained-release part is 1:3-1:2; in some embodiments, the weight ratio of oseltamivir in the immediate-release part and in the sustained-release part is 1:3-1:1; in some embodiments, the weight ratio of oseltamivir in the immediate-release part and in the sustained-release part is 1:2-1:1. The formulation can be a biphasic release formulation, a three-phase release formulation, or a multiphase release formulation with more than three phases. In some embodiments, the formulation is a biphasic release formulation, in some embodiments, the formulation is a three-phase release formulation, and in some embodiments, the formulation is a multiphase release formulation. In some embodiments, the active ingredient is oseltamivir; in some embodiments, the active ingredient is oseltamivir phosphate.

In some embodiments, the oseltamivir formulation comprises at least one sustained-release material, and the weight ratio of oseltamivir is 3%-50% of the total weight of the formulation; the release amount of oseltamivir or salt thereof at 4 h is 25%-90%, and the release amount of oseltamivir or salt thereof at 10 h is greater than 70%. In some embodiments, the sustained-release materials in the oseltamivir formulation are methacrylic acid-ethyl acrylate copolymer RL and methacrylic acid-ethyl acrylate copolymer RS, and the weight ratio of oseltamivir is 22.4%, the release amount of oseltamivir at 4 h is 78%, the release amount of oseltamivir or salt thereof at 10 h is 97%; in some embodiments, the sustained-release material in the oseltamivir formulation is ethyl cellulose, and the weight ratio of seltamivir is 3.3%, the release amount of oseltamivir at 4 h is 75%, and the release amount of oseltamivir or salt thereof at 10 h is 96%.

In some embodiments, in the oseltamivir formulation, the release amount of oseltamivir or salt thereof at 1 h is 25%-55%, the release amount of oseltamivir or salt thereof at 4 h is 25%-90%, the release amount of oseltamivir or salt thereof at 10 h is greater than 70%; the weight ratio of oseltamivir or salt thereof is 3%-50%; according to the weight of oseltamivir, the single-dose strength is 60 mg-300 mg; after administration once a day, the peak-to-valley ratio of the plasma concentration in vivo of the oseltamivir active metabolite within 24 h is less than 2.5:1. Optionally, in some embodiments, the oseltamivir formulation further comprises at least one sustained-release material, and the weight ratio of the sustained-release material is 3%-50%, and the sustained-release material includes at least one selected from ethyl cellulose, hydroxypropyl methyl cellulose, cellulose acetate, polyoxyethylene, polyvinyl alcohol, methacrylic acid-ethyl acrylate copolymer RL, methacrylic acid-ethyl acrylate copolymer RS, methacrylic acid-ethyl acrylate copolymer NE 30D, methacrylic acid-ethyl acrylate copolymer L100-55, hypromellose acetate succinate, glyceryl behenate, chitosan, carbomer, carnauba wax, Kollicoat® SR 30D, Kollidon® SR, cellulose acetate titanate, sodium alginate, sodium carboxymethyl cellulose, carrageenan, cetyl alcohol, stearyl alcohol, cetostearyl alcohol and paraffin.

The sixth aspect of the present invention provides an oseltamivir formulation, comprising oseltamivir or salt thereof, the formulation is administered once a day, and the formulation comprises a sustained-release part containing oseltamivir or salt thereof and an immediate-release part containing oseltamivir or salt thereof. According to the total weight of oseltamivir in the formulation, the weight ratio of oseltamivir in the immediate-release part is 20%-50%. The formulation comprises at least one sustained-release material, and the weight ratio of the sustained-release material is 3%-50%; the release amount of oseltamivir at 1 h is 25%-55%, and the release amount at 4 h is 25%-90%, the release amount at 10 h is greater than 70%.

In some embodiments, for the oseltamivir formulation, the weight ratio of oseltamivir in the immediate-release part and in the sustained-release part is 1:4-1:1. In some embodiments, the weight ratio of oseltamivir in the immediate-release part and in the sustained-release part is 1:4-1:3; in some embodiments, the weight ratio of oseltamivir in the immediate-release part and in the sustained-release part is 1:4-1:2; in some embodiments, the weight ratio of oseltamivir in the immediate-release part and in the sustained-release part is 1:3-1:2; in some embodiments, the weight ratio of oseltamivir in the immediate-release part and in the sustained-release part is 1:3-1:1; in some embodiments, the weight ratio of oseltamivir in the immediate-release part and in the sustained-release part is 1:2-1:1; in some embodiments, the weight ratio of oseltamivir in the immediate-release part and in the sustained-release part is 1:4, 1:3, 1:2 or 1:1.

According to the weight of oseltamivir, the single-dose strength of the oseltamivir formulation is 60 mg-300 mg; in some embodiments, the strength is 60 mg-90 mg; in some embodiments, the strength is 60 mg-150 mg; in some embodiments, the strength is 60 mg-200 mg; in some embodiments, the strength is 90 mg-150 mg; in some embodiments, the strength is 90 mg-200 mg; in some embodiments, the strength is 90 mg-300 mg; in some embodiments, the strength is 150 mg-200 mg; in some embodiments, the strength is 150 mg-300 mg; in some embodiments, the strength is 200 mg-300 mg. In some embodiments, the strength is 60 mg, 90 mg, 150 mg, 200 mg or 300 mg. The current marketed immediate-release dosage form for children, according to the weight of oseltamivir, has a minimum strength of 30 mg, administered twice a day. If it is prepared as a sustained-release formulation administered once a day, the minimum strength is 60 mg. The currently marketed adult immediate-release dosage form, according to the weight of oseltamivir, the minimum strength is 75 mg, administered twice a day. If it is prepared as a sustained-release formulation administered once a day, the minimum strength is 150 mg, if the strength is increased to 300 mg, the effect cannot be equivalent with the immediate-release dosage form of 75 mg strength and administered twice a day, indicating that the bioavailability is low. If there is a burst release, the risk of toxic and side effects will increase, and the tablet is heavy, causing difficulty in swallowing. Therefore, according to the weight of oseltamivir, 60 mg-300 mg is a more appropriate strength.

In some embodiments, the sustained-release material comprises at least one selected from ethyl cellulose, hydroxypropyl methyl cellulose, cellulose acetate, polyoxyethylene, polyvinyl alcohol, methacrylic acid-ethyl acrylate copolymer RL, methacrylic acid-ethyl acrylate copolymer RS, methacrylic acid-ethyl acrylate copolymer NE 30D, methacrylic acid-ethyl acrylate copolymer L100-55, hypromellose acetate succinate, glyceryl behenate, chitosan, carbomer, carnauba wax, Kollicoat® SR 30D, Kollidon® SR, cellulose acetate titanate, sodium alginate, sodium carboxymethyl cellulose, carrageenan, cetyl alcohol, stearyl alcohol, cetostearyl alcohol and paraffin.

The oseltamivir formulation is used for the treatment of adult influenza A or adult influenza B, as well as childhood influenza A or childhood influenza B, and after administration once a day, the plasma concentration in vivo of the oseltamivir active metabolite within 24 h is greater than 100 ng/ml, or greater than 150 ng/ml. In some embodiments, the plasma concentration in vivo of the oseltamivir active metabolite within 24 h is greater than 172 ng/ml; in some embodiments, the plasma concentration in vivo of the oseltamivir active metabolite within 24 h is greater than 261 ng/ml.

The oseltamivir formulation, after administration once a day, the peak-to-valley ratio of the plasma concentration in vivo of the oseltamivir active metabolite within 24 h is less than 2.5:1. The peak-to-valley ratio of plasma concentration in vivo is less than 2.5:1, which can avoid peak-to-valley fluctuations, thereby improving the treatment compliance and safety of patients. In some embodiments, the peak-to-valley ratio of the plasma concentration in vivo of the oseltamivir active metabolite within 24 h is 2.14:1. In some embodiments, the oseltamivir formulation, after administration once a day, the peak-to-valley ratio of the plasma concentration in vivo of the oseltamivir active metabolite within 24 h is less than 2:1, which is more conducive to avoiding peak-to-valley fluctuations, thereby improving the treatment compliance and safety of patients. In some embodiments, the peak-to-valley ratio of the plasma concentration in vivo of the oseltamivir active metabolite within 24 h is 1.21:1.

For the oseltamivir formulation, after administration once a day, the plasma concentration in vivo of the oseltamivir active metabolite at 2 h is more than 20%, or more than 30%, or more than 40% of Cmax; the plasma concentration in vivo at 24 h is more than 30%, or more than 40% of Cmax. In some embodiments, the plasma concentration in vivo of the oseltamivir active metabolite at 2 h is 27% of Cmax; in some embodiments, the plasma concentration in vivo of the oseltamivir active metabolite at 2 h is 25% of Cmax; in some embodiments, the plasma concentration in vivo of the oseltamivir active metabolite at 24 h is 47% of Cmax; in some embodiments, the plasma concentration in vivo of the oseltamivir active metabolite at 24 h is 82% of Cmax.

The oseltamivir formulation can continuously release oseltamivir for a period of at least 24 h.

In the oseltamivir formulation, the plasma concentration in vivo of the oseltamivir active metabolite is greater than or equal to 100 ng/ml, and the maintenance time is greater than 16 h, or greater than 20 h. In some embodiments, for the oseltamivir formulation, the plasma concentration in vivo of the oseltamivir active metabolite is greater than or equal to 170 ng/ml, and the maintenance time is greater than 16 h. In some embodiments, for the oseltamivir formulation, the plasma concentration in vivo of the oseltamivir active metabolite is greater than or equal to 250 ng/ml, and the maintenance time is greater than 12 h. In some embodiments, for the oseltamivir formulation, the plasma concentration in vivo of the oseltamivir active metabolite is greater than or equal to 250 ng/ml, and the maintenance time is greater than 16 h. In some embodiments, the plasma concentration in vivo of the oseltamivir active metabolite is 261 ng/ml, and the maintenance time is 16 h; in some embodiments, the plasma concentration in vivo of the oseltamivir active metabolite is 172 ng/ml, and the maintenance time is 20 h.

The seventh aspect of the present invention provides an oseltamivir formulation, further comprising a sustained-release material, the sustained-release material comprises hydroxypropyl methylcellulose and/or methacrylic acid-ethyl acrylate copolymer. In some embodiments, the sustained-release material comprises hydroxypropyl methylcellulose; in some embodiments, the sustained-release material comprises methacrylic acid-ethyl acrylate copolymer; in some embodiments, the sustained-release material comprises hydroxypropyl methylcellulose and methacrylic acid-ethyl acrylate copolymer.

The methacrylic acid-ethyl acrylate copolymer comprises at least one selected from methacrylic acid-ethyl acrylate copolymer RL, methacrylic acid-ethyl acrylate copolymer RS, methacrylic acid-ethyl acrylate copolymer NE 30D, methacrylic acid-ethyl acrylate copolymer L100-55. In some embodiments, the methacrylic acid-ethyl acrylate copolymer comprises methacrylic acid-ethyl acrylate copolymer RL; in some embodiments, the methacrylic acid-ethyl acrylate copolymer comprises methacrylic acid-ethyl acrylate copolymer RS; in some embodiments, the methacrylic acid-ethyl acrylate copolymer comprises methacrylic acid-ethyl acrylate copolymer NE 30D; in some embodiments, the methacrylic acid-ethyl acrylate copolymer comprises methacrylic acid-ethyl acrylate copolymer L100-55.

The hydroxypropyl methyl cellulose comprises at least one selected from hydroxypropyl methyl cellulose K15M, hydroxypropyl methyl cellulose K4M, hydroxypropyl methyl cellulose K100M, hydroxypropyl methyl cellulose K100LV. In some embodiments, the hydroxypropyl methyl cellulose comprises hydroxypropyl methyl cellulose K15M; in some embodiments, the hydroxypropyl methyl cellulose comprises hydroxypropyl methyl cellulose K4M; in some embodiments, the hydroxypropyl methyl cellulose comprises hydroxypropyl methyl cellulose K100M; in some embodiments, the hydroxypropyl methyl cellulose comprises hydroxypropyl methyl cellulose K100LV.

In some embodiments, the weight ratio of the sustained-release material is 3%-50% of the total weight of the formulation. In some embodiments, the weight ratio of the sustained-release material is 3%-10%; in some embodiments, the weight ratio of the sustained-release material is 3%-20%; in some embodiments, the weight ratio of the sustained-release material is 3%-30%; in some embodiments, the weight ratio of the sustained-release material is 10%-20%; in some embodiments, the weight ratio of the sustained-release material is 10-30%; in some embodiments, the weight ratio of the sustained-release material is 10%-50%; in some embodiments, the weight ratio of the sustained-release material is 20%-30%; in some embodiments, the weight ratio of the sustained-release material is 20%-50%; in some embodiments, the weight ratio of the sustained-release material is 30%-50%. In some embodiments, the weight ratio of the sustained-release material is 37%, 29%, 25%, 50%, 21% or 34%.

In some embodiments, the weight ratio of the hydroxypropyl methyl cellulose is 4%-30% of the total weight of the formulation, and the weight ratio of the methacrylic acid-ethyl acrylate copolymer is 0-35% of the total weight of the formulation. In some embodiments, the weight ratio of the hydroxypropyl methyl cellulose is 4%40%, in some embodiments, the weight ratio of the hydroxypropyl methyl cellulose is 4%-15%, in some embodiments, the weight ratio of the hydroxypropyl methyl cellulose is 4%-20%, in some embodiments, the weight ratio of the hydroxypropyl methyl cellulose is 4%-25%, in some embodiments, the weight ratio of the hydroxypropyl methyl cellulose is 10%45%, in some embodiments, the weight ratio of the hydroxypropyl methyl cellulose is 10%-20%, in some embodiments, the weight ratio of the hydroxypropyl methyl cellulose is 10%-25%, in some embodiments, the weight ratio of the hydroxypropyl methyl cellulose is 10%-30%, in some embodiments, the weight ratio of the hydroxypropyl methyl cellulose is 15%-20%, in some embodiments, the weight ratio of the hydroxypropyl methyl cellulose is 15%-25%, in some embodiments, the weight ratio of the hydroxypropyl methyl cellulose is 15%-30%, in some embodiments, the weight ratio of the hydroxypropyl methyl cellulose is 20%-25%, in some embodiments, the weight ratio of the hydroxypropyl methyl cellulose is 20%-30%; in some embodiments, the weight ratio of the methacrylic acid-ethyl acrylate copolymer is 5%40%, in some embodiments, the weight ratio of the methacrylic acid-ethyl acrylate copolymer is 5%-20%, in some embodiments, the weight ratio of the methacrylic acid-ethyl acrylate copolymer is 5%-25%, in some embodiments, the weight ratio of the methacrylic acid-ethyl acrylate copolymer is 5%-35%, in some embodiments, the weight ratio of the methacrylic acid-ethyl acrylate copolymer is 10%-20%, in some embodiments, the weight ratio of the methacrylic acid-ethyl acrylate copolymer is 10%-25%, in some embodiments, the weight ratio of the methacrylic acid-ethyl acrylate copolymer is 10%-35%, in some embodiments, the weight ratio of the methacrylic acid-ethyl acrylate copolymer is 20%-25%, in some embodiments, the weight ratio of the methacrylic acid-ethyl acrylate copolymer is 20%-35%, in some embodiments, the weight ratio of the methacrylic acid-ethyl acrylate copolymer is 25%-35%. In some embodiments, the weight ratio of the hydroxypropyl methyl cellulose is 12%, the weight ratio of the methacrylic acid-ethyl acrylate copolymer is 25%; in some embodiments, the weight ratio of the hydroxypropyl methyl cellulose is 4%, the weight ratio of the methacrylic acid-ethyl acrylate copolymer is 25%; in some embodiments, the weight ratio of the hydroxypropyl methyl cellulose is 12%, the weight ratio of the methacrylic acid-ethyl acrylate copolymer is 12%; in some embodiments, the weight ratio of the hydroxypropyl methyl cellulose is 17%, the weight ratio of the methacrylic acid-ethyl acrylate copolymer is 33%; in some embodiments, the weight ratio of the hydroxypropyl methyl cellulose is 8%, the weight ratio of the methacrylic acid-ethyl acrylate copolymer is 12%; in some embodiments, the weight ratio of the hydroxypropyl methyl cellulose is 25%, the weight ratio of the methacrylic acid-ethyl acrylate copolymer is 4%; in some embodiments, the weight ratio of the hydroxypropyl methyl cellulose is 11%, the weight ratio of the methacrylic acid-ethyl acrylate copolymer is 23%.

In some embodiments, the oseltamivir formulation further comprises a sustained-release material, the sustained-release material is hydroxypropyl methyl cellulose and methacrylic acid-ethyl acrylate copolymer, and the weight ratio of the sustained-release material is 3%-50%, or 20%-50%, or 30%-50%, or 10%-30% of the total weight of the formulation; the weight ratio of hydroxypropyl methyl cellulose is 4%-30%, or 4%-15%, or 10%-20%, or 15%-30% of the total weight of the formulation; the weight ratio of methacrylic acid-ethyl acrylate copolymer is 0-35%, or 5%-25%, or 10%-30%, or 20%-35% of the total weight of the formulation.

In some embodiments, for the oseltamivir formulation, the release amount of oseltamivir within 1 h is 25%-55%; the release amount of oseltamivir within 4 h is 25%-90%; the release amount of oseltamivir within 10 h is greater than 70%; the weight ratio of oseltamivir is 3%-50% of the total weight of the formulation; the formulation also comprises at least one sustained-release material, the weight ratio of the sustained-release material is 3%-50% of the total weight of the formulation. After administration once a day, the peak-to-valley ratio of the plasma concentration in vivo of the oseltamivir active metabolite within 24 h is less than 2.5:1; the oseltamivir formulation is used for the treatment of influenza A or influenza B in adults, influenza A or influenza B in children, after administration once a day, the plasma concentration in vivo of the oseltamivir active metabolite within 24 h is greater than 100 ng/mL; after administration of the oseltamivir formulation once a day, the plasma concentration in vivo of the oseltamivir active metabolite within 2 h is more than 20% of Cmax, and the plasma concentration in vivo within 24 h is more than 30% of Cmax.

The oseltamivir formulation provided in the first to seventh aspects of the present invention can be prepared into a tablet, a capsule or a suspension. In some embodiments, the oseltamivir formulation is a tablet; in some embodiments, the oseltamivir formulation is a capsule; in some embodiments, the oseltamivir formulation is a suspension.

The oseltamivir formulation provided in the first to seventh aspects of the present invention can be prepared into a double-layer tablet, wherein one layer of the double-layer tablet comprises an immediate-release part and the other layer comprises a sustained-release part.

The oseltamivir formulation provided by the first to seventh aspects of the present invention takes the sustained-release part as the tablet core, and the immediate-release part is wrapped outside the tablet core.

The oseltamivir formulation provided by the first to seventh aspects of the present invention comprises a sustained-release part and an immediate-release part, the sustained-release part comprises bead 1, and the immediate-release part comprises bead 2.

The bead 1 can be prepared by uniformly mixing oseltamivir and the matrix sustained-release material, and the bead 2 can be prepared by uniformly mixing oseltamivir and the matrix material.

The bead 1 may contain oseltamivir and the exterior is coated with a sustained-release coating layer formed by high polymer and other pharmaceutical excipients, and the bead 2 may contain oseltamivir and the exterior is coated with a immediate-release coating layer formed by water-soluble polymer film-forming material and plasticizer.

The bead 1 can be prepared by the extrusion spheronization method after mixing oseltamivir with matrix sustained-release material, and the bead 2 can be prepared by the extrusion spheronization method after mixing oseltamivir with pharmaceutical excipients.

The bead 1 can be prepared by spraying oseltamivir and the sustained-release film-forming material on a blank pellet, the bead 2 can be prepared by spraying oseltamivir and the immediate-release coating material on a blank pellet.

In some embodiments, the bead 1 and the bead 2 are mixed and compressed into a double-layer or multi-layer tablet, or the bead 1 and the bead 2 are mixed and directly filled in capsules, or the bead 1 and the bead 2 are mixed and then bagged.

The eighth aspect of the present invention provides the preparation method of the aforementioned oseltamivir formulation. A preparation method of oseltamivir formulation as described in the first to the seventh aspect, comprising the following steps:

(1) the immediate-release coating layer formed by water-soluble polymer film-forming material, plasticizer and oseltamivir is wrapped around the blank core to obtain the immediate-release part,

(2) the sustained-release coating layer formed by high polymer and other pharmaceutical excipients is wrapped around the immediate-release part obtained in step (1) to obtain the sustained-release part,

(3) filling the immediate-release part obtained in step (1) and the sustained-release part obtained in step (2) into capsules, or compressing into tablets, or bagging to obtain the oseltamivir formulation.

A preparation method of oseltamivir formulation as described in the first to the seventh aspect, comprising the following steps:

(1) the sustained-release coating layer formed by high polymer and other pharmaceutical excipients is wrapped around the bead containing oseltamivir to obtain a sustained-release part,

(2) the immediate-release coating layer formed by water-soluble polymer film-forming material, plasticizer and oseltamivir is wrapped around the sustained-release part obtained in step (1) to obtain the oseltamivir formulation.

A preparation method of oseltamivir formulation as described in the first to the seventh aspect, comprising the following steps:

(1) the sustained-release coating layer formed by oseltamivir, high polymer and other pharmaceutical excipients is wrapped around a blank core to obtain the sustained-release part,

(2) the immediate-release coating layer formed by water-soluble polymer film-forming material, plasticizer and oseltamivir is wrapped around the sustained-release part obtained in step (1) to obtain the oseltamivir formulation.

A preparation method of oseltamivir formulation as described in the first to the seventh aspect, comprising the following steps:

(1) the sustained-release part is the core containing oseltamivir and matrix sustained-release material,

(2) the immediate-release coating layer formed by water-soluble polymer film-forming material, plasticizer and oseltamivir is wrapped around the sustained-release part obtained in step (1) to obtain the oseltamivir formulation.

In the preparation method provided herein, the bead may comprise granules, tablet core or pellet.

In the preparation method provided herein, the bead may further comprise, optionally, a filler, a binder, a penetration enhancer or a lubricant.

In the preparation method provided herein, the method for preparing the bead comprises wet granulation, dry granulation or direct powder compression.

In the preparation method provided herein, the high polymer may comprise at least one selected from ethyl cellulose, cellulose acetate, methacrylic acid-ethyl acrylate copolymer RL, methacrylic acid-ethyl acrylate copolymer RS, methacrylic acid-ethyl acrylate copolymer NE 30D, Kollicoat® SR 30D, Kollidon® SR, methacrylic acid-ethyl acrylate copolymer L100-55, hypromellose acetate succinate, cellulose acetate titanate.

In the preparation method provided herein, the polymer comprises a pH-independent polymer material or a pH-dependent polymer material. The pH-independent polymer material includes at least one selected from ethyl cellulose, hydroxypropyl methyl cellulose, cellulose acetate, polyoxyethylene, polyvinyl alcohol, methacrylic acid-ethyl acrylate copolymer RL, methacrylic acid-ethyl acrylate copolymer RS, methacrylic acid-ethyl acrylate copolymer NE 30D, Kollicoat® SR 30D, Kollidon® SR; the pH-dependent polymer material includes at least one selected from methacrylic acid-ethyl acrylate copolymer L100-55, hypromellose acetate succinate, cellulose acetate titanate, chitosan, carbomer, carrageenan, sodium alginate, sodium carboxymethyl cellulose.

In the preparation method provided herein, the other pharmaceutical excipients may include plasticizers, anti-sticking agents, emulsifiers, or porogens.

In the preparation method provided herein, the water-soluble polymer film-forming material may include at least one selected from hydroxypropyl methyl cellulose, polyvinylpyrrolidone, polyvinyl alcohol, hydroxypropyl cellulose, hydroxyethyl cellulose, hydroxyethyl methyl cellulose, copovidone.

In the preparation method provided herein, the matrix sustained-release material may include at least one selected from hydroxypropyl methyl cellulose, polyoxyethylene, polyvinyl alcohol, ethylcellulose, glyceryl behenate, chitosan, carbomer, carnauba wax, sodium alginate, sodium carboxymethyl cellulose, carrageenan, cetyl alcohol, stearyl alcohol, cetostearyl alcohol, paraffin.

In the preparation method provided herein, the matrix sustained-release material includes a pH-independent polymer material, a pH-dependent polymer material or a waxy matrix material. The pH-independent polymer material includes at least one selected from ethyl cellulose, hydroxypropyl methylcellulose, polyoxyethylene, and polyvinyl alcohol; the pH-dependent polymer material includes at least one selected from thitosan, tarbomer, tarrageenan, sodium alginate, sodium carboxymethyl cellulose; the waxy matrix material includes at least one selected from glyceryl behenate, carnauba wax, cetyl alcohol, stearyl alcohol, cetostearyl alcohol, paraffin.

In the preparation method provided herein, the immediate-release coating layer is externally wrapped with an isolation layer.

In some embodiments, the isolation layer includes a water-soluble polymer film-forming material, or a plasticizer, and optionally, an anti-sticking agent or an opacifier.

### DESCRIPTION OF THE DRAWINGS

FIG. 1 is the drug-time curve of the oseltamivir active metabolite in vivo after oral administration of the oseltamivir formulation prepared in Example 12 of the present invention in an adult's fasting state.

FIG. 2 is the drug-time curve of the oseltamivir active metabolite in vivo after oral administration of the oseltamivir formulation prepared in Example 12 of the present invention in an adult's feeding state.

FIG. 3 is the drug-time curve of the oseltamivir active metabolite in vivo after oral administration of the oseltamivir formulation prepared in Example 18 of the present invention in an adult's feeding state.

### EXAMPLES

### Example 1

Formulation Form
150 mg oseltamivir sustained-release formulation

| | Component | Formulation ratio (%) |
|---|---|---|
| Tablet core | Oseltamivir phosphate | 40.42 |
| | Microcrystalline cellulose | 39.65 |
| | Polyvinylpyrrolidone | 2.05 |
| | Sodium chloride | 5.00 |
| | Microcrystalline cellulose | 8.88 |
| | Micro powder silica | 1.00 |
| | Sodium stearate fumarate | 3.00 |
| | Total | 100.00 |
| Controlled- | Cellulose acetate | 6.00 |
| release film | Polyethylene glycol 6000 | 1.50 |
| | Purified water | 6.00 |
| | acetone | 86.50 |
| | Total | 100.00 |
| Immediate- | Oseltamivir phosphate | 7.88 |
| release layer | Hydroxypropyl methylcellulose E5 | 4.00 |
| | Polyethylene glycol 400 | 2.00 |
| | Purified water | 86.21 |
| | Total | 100.00 |
| Isolation | Opadry 85F | 15.00 |
| layer | Purified water | 85.00 |
| | Total | 100.00 |

Preparation of tablet core: oseltamivir phosphate and microcrystalline cellulose were weighed and transferred to a granulator for premixing, and polyvinylpyrrolidone-water was used as granulation solution for wet granulation (the amount of the final dried polyvinylpyrrolidone is shown in the formulation), then wet granulated, fluidized bed drying, dry granulated in sequence, and then filler, penetration enhancer and lubricant were added, the mixture was pressed into tablet to obtain a 100 mg oseltamivir tablet core.

Controlled-release film: the controlled-release film coating solution was prepared and used to coat the above-mentioned tablet core, the percentage of coating weight gain in the tablet core was 8%.

Immediate-release layer: the immediate-release layer coating solution was prepared and used to coat the above-mentioned controlled-release coated tablet, and the coating film contains 50 mg of oseltamivir.

Isolation layer: the isolation layer coating solution was prepared and used to coat the above-mentioned immediate-release coated tablet, and the percentage of coating weight gain in the immediate-release coated tablet was 2%.

### Example 2

Formulation Form
60 mg oseltamivir sustained-release formulation

| | Component | Formulation ratio (%) |
|---|---|---|
| Tablet core | Oseltamivir phosphate | 42.04 |
| | Microcrystalline cellulose 101QD | 36.81 |
| | Polyvinylpyrrolidone | 2.15 |
| | Sodium chloride | 5.00 |
| | Microcrystalline cellulose PH102 | 10.00 |
| | Micro powder silica | 1.00 |
| | Sodium stearate fumarate | 3.00 |
| | Total | 100.00 |
| Controlled- | Methacrylic acid-ethyl acrylate | 2.00 |
| release film | copolymer RL | |
| | Methacrylic acid-ethyl acrylate | 8.00 |
| | copolymer RS | |
| | Tween 80 | 0.20 |
| | Triethyl citrate | 2.00 |
| | Glyceryl Monostearate | 0.50 |
| | Purified water | 87.30 |
| | Total | 100.00 |
| Immediate- | Oseltamivir phosphate | 7.88 |
| release layer | Hydroxypropyl methylcellulose E5 | 4.00 |
| | Polyethylene glycol 400 | 2.00 |
| | Purified water | 86.12 |
| | Total | 100.00 |
| Isolation | Opadry 85F | 15.00 |
| layer | Purified water | 85.00 |
| | Total | 100.00 |

Preparation of tablet core: oseltamivir phosphate and microcrystalline cellulose 101QD were weighed and transferred to a granulator for premixing, and polyvinylpyrrolidone-water was used as granulation solution for wet granulation (the amount of the final dried polyvinylpyrrolidone is shown in the formulation), then wet granulated, fluidized bed drying, dry granulated in sequence, and then filler, penetration enhancer and lubricant were added, the mixture was pressed into tablet to obtain a 40 mg oseltamivir tablet core.

Controlled-release film 1: the controlled-release film coating solution was prepared and used to coat the above-mentioned tablet core, the percentage of coating weight gain in the tablet core was 10%.

Immediate-release layer: the immediate-release layer coating solution was prepared and used to coat the above-mentioned controlled-release coated tablet, and the coating film contains 20 mg of oseltamivir.

Isolation layer: the isolation layer coating solution was prepared and used to coat the above-mentioned immediate-release coated tablet, and the percentage of coating weight gain in the immediate-release coated tablet was 2%.

### Example 3

Formulation Form
180 mg oseltamivir sustained-release formulation

| | Component | Formulation ratio (%) |
|---|---|---|
| Tablet core | Oseltamivir phosphate | 41.15 |
| | Mannitol | 29.52 |
| | Polyvinylpyrrolidone | 1.23 |
| | Microcrystalline cellulose | 16.60 |
| | Sodium chloride | 7.50 |
| | Micro powder silica | 1.00 |
| | Sodium stearate fumarate | 3.00 |
| | Total | 100.00 |
| Controlled-release | Ethyl cellulose | 8.00 |
| film | Polyethylene glycol 4000 | 4.00 |
| | Triethyl citrate | 2.00 |
| | ethanol | 86.00 |
| | Total | 100.00 |
| Immediate-release | Oseltamivir phosphate | 7.88 |
| layer | Hydroxypropyl | 4.00 |
| | methylcellulose E5 | |
| | Polyethylene glycol 400 | 2.00 |
| | Purified water | 86.21 |
| | Total | 100.00 |
| Isolation layer | Opadry 85F | 15.00 |
| | Purified water | 85.00 |

Preparation of tablet core: oseltamivir phosphate and mannitol were weighed and transferred to a granulator for premixing, and polyvinylpyrrolidone-water was used as granulation solution for wet granulation (the amount of final dried polyvinylpyrrolidone is shown in the formulation), then wet granulated, fluidized bed drying, dry granulated in sequence, and then filler, penetration enhancer and lubricant were added, the mixture was pressed into tablet to obtain a 130 mg oseltamivir tablet core.

Controlled-release film: the controlled-release film coating solution was prepared and used to coat the above-mentioned tablet core, the percentage of coating weight gain in the tablet core was 8%.

Immediate-release layer: the immediate-release layer coating solution was prepared and used to coat the above-mentioned controlled-release coated tablet, and the coating film contains 50 mg of oseltamivir.

Isolation layer: the isolation layer coating solution was prepared and used to coat the above-mentioned immediate-release coated tablet, and the percentage of coating weight gain in the immediate-release coated tablet was 2%.

### Example 4

Formulation Form
90 mg oseltamivir sustained-release formulation

| | Component | Formulation ratio (%) |
|---|---|---|
| Sustained-release | Oseltamivir phosphate | 30.32 |
| tablet core | Hydroxypropyl methyl | 64.83 |
| | cellulose K100 LV | |
| | Polyvinylpyrrolidone | 0.85 |
| | Micro powder silica | 1.00 |
| | Sodium stearyl fumarate | 3.00 |
| | Total | 100.00 |
| Immediate-release | Oseltamivir phosphate | 7.88 |
| layer | Hydroxypropyl | 4.00 |
| | methylcellulose E5 | |
| | Polyethylene glycol 400 | 2.00 |
| | Purified water | 86.21 |
| | Total | 100.00 |
| Isolation layer | Opadry 85F | 15.00 |
| | Purified water | 85.00 |
| | Total | 100.00 |

Preparation of tablet core: oseltamivir phosphate and hydroxypropyl methylcellulose K100 LV were weighed and transferred to a granulator for premixing, and polyvinylpyrrolidone-water was used as granulation solution for wet granulation (the amount of the final dried polyvinylpyrrolidone is shown in the formulation), then wet granulated, fluidized bed drying, and dry granulated in sequence, then lubricant was added and the mixture was pressed into tablet to obtain a 60 mg oseltamivir sustained-release tablet core.

Immediate-release layer: the immediate-release layer coating solution was prepared and used to coat the above-mentioned sustained-release tablet core, and the coating film contains 30 mg of oseltamivir.

Isolation layer: the isolation layer coating solution was prepared and used to coat the above-mentioned immediate-release coated tablet, and the percentage of coating weight gain in the immediate-release coated tablet was 2%.

### Example 5

Formulation Form
150 mg oseltamivir sustained-release formulation

| | Component | Formulation ratio (%) |
|---|---|---|
| Sustained-release | Oseltamivir phosphate | 41.04 |
| layer | Microcrystalline cellulose | 10.46 |
| | PH101 | |
| | Hydroxypropyl methyl | 10.00 |
| | cellulose K4M | |
| | Hydroxypropyl methyl | 30.00 |
| | cellulose K100 LV | |
| | Polyvinylpyrrolidone | 2.50 |
| | Micro powder silica | 1.00 |
| | Sodium stearyl fumarate | 5.00 |
| | Total | 100.00 |
| Immediate-release | Oseltamivir phosphate | 43.79 |
| layer | Microcrystalline cellulose | 39.21 |
| | Polyvinylpyrrolidone | 3.00 |
| | Sodium carboxymethyl | 10.00 |
| | starch | |
| | Micro powder silica | 1.00 |
| | Sodium stearyl fumarate | 3.00 |
| | Total | 100.00 |
| Isolation layer | Opadry 85F | 15.00 |
| | Purified water | 85.00 |
| | Total | 100.00 |

### Example 6

Formulation Form
200 mg sustained-release formulation of oseltamivir

| | Component | Formulation ratio (%) |
|---|---|---|
| Sustained-release | Oseltamivir phosphate | 39.41 |
| layer | Microcrystalline cellulose | 32.06 |
| | Hydroxypropyl methyl | 20.00 |
| | cellulose K100 LV | |
| | Polyvinylpyrrolidone | 2.53 |
| | Micro powder silica | 1.00 |
| | Sodium stearyl fumarate | 5.00 |
| | Total | 100.00 |
| Immediate-release | Oseltamivir phosphate | 43.79 |
| layer | Microcrystalline cellulose | 39.21 |
| | Polyvinylpyrrolidone | 3.00 |
| | Sodium carboxymethyl | 10.00 |
| | starch | |
| | Micro powder silica | 1.00 |
| | Sodium stearyl fumarate | 3.00 |
| | Total | 100.00 |
| Isolation layer | Opadry 85F | 15.00 |
| | Purified water | 85.00 |
| | Total | 100.00 |

### Preparation Method of Example 5 and Example 6

Preparation of sustained-release layer bulk granules: oseltamivir phosphate, microcrystalline cellulose and hydroxypropyl methyl cellulose were weighed and transferred to a granulator for premixing, and polyvinylpyrrolidone-water was used as granulation solution for wet granulation (the amount of the final dried polyvinylpyrrolidone is shown in the formulation), then wet granulated, fluidized bed drying, dry granulated in sequence, and then lubricant was added to obtain the sustained-release layer bulk granules.

Preparation of immediate-release layer bulk granules: oseltamivir phosphate and microcrystalline cellulose were weighed and transferred to a granulator for premixing, and polyvinylpyrrolidone-water was used as granulation solution for wet granulation (the amount of the final dried polyvinylpyrrolidone is shown in the formulation), and then wet granulated, fluidized bed drying, dry granulated in sequence, and then lubricant was added to obtain the immediate-release layer bulk granules.

Preparation of double-layer tablet: a double-layer tablet press was used to fill 100 mg or 150 mg of sustained-release layer bulk granules, then pre-pressed to obtain the sustained-release layer tablet core; then 50 mg of immediate-release layer granules were filled and pressed into tablet to obtain the immediate-release and sustained-release double-layered tablet core.

Isolation layer: an isolation layer coating solution was prepared and used to coat the above-mentioned immediate-release and sustained-release double-layered tablet core, and the percentage of coating weight gain in the tablet core was 2%.

### Example 7

Formulation Form
10 mg immediate-release granules

| Formulation component | Formulation ratio (%) |
|---|---|
| Oseltamivir phosphate | 8.54 |
| Sucrose | 90.59 |
| Polyvinylpyrrolidone | 0.87 |
| Total | 100.00 |

50 mg sustained-release granules

| | Formulation component | Formulation ratio (%) |
|---|---|---|
| Immediate-release | Oseltamivir phosphate | 8.54 |
| granules | Microcrystalline | 90.59 |
| | cellulose | |
| | Polyvinylpyrrolidone | 0.87 |
| | Total | 100.00 |
| Sustained-release | Ethyl cellulose | 8.00 |
| layer | Polyethylene glycol 6000 | 4.00 |
| | Triethyl citrate | 2.00 |
| | Purified water | 6.00 |
| | ethanol | 80.00 |
| | Total | 100.00 |

| | |
|---|---|
| Formulation component | Formulation ratio (%) |

Blank granules

| | |
|---|---|
| Sucrose | 97.41 |
| Xanthan Gum | 1.00 |
| Sodium citrate dihydrate | 0.50 |
| Sodium benzoate | 0.25 |
| Polyvinylpyrrolidone | 0.84 |
| Total | 100.00 |

60 mg oseltamivir sustained-release formulation

| | |
|---|---|
| 10 mg immediate-release granules | 3.25 |
| 50 mg sustained-release granules | 21.94 |
| Blank granules | 71.06 |
| Sucralose | 2.00 |
| Orange essence | 0.75 |
| Micro powder silica | 1.00 |
| Total | 100.00 |

Preparation of immediate-release granules: oseltamivir phosphate and sucrose were weighed and transferred to a granulator for premixing, and polyvinylpyrrolidone-water was used as granulation solution for wet granulation (the amount of the final dried polyvinylpyrrolidone is shown in the formulation), and then wet granulated, fluidized bed drying, and dry granulated in sequence to obtain the immediate-release granules.

Preparation of sustained-release granules: sustained-release coating solution was prepared, then immediate-release granules were taken and sustained-release coated, the percentage of coating weight gain in the granules was 35%.

Preparation of blank granules: all components (except polyvinylpyrrolidone) were weighed and transferred to a granulator for premixing, and polyvinylpyrrolidone-water was used as granulation solution for wet granulation (the amount of the final dried polyvinylpyrrolidone is shown in the formulation), and then wet granulated, fluidized bed drying, and dry granulated in sequence to obtain blank granules.

Preparation of total bulk granules: 10 mg of immediate-release granules, 50 mg of sustained-release granules, blank granules were weighed, then flavoring and glidant were added, and the mixture was mixed to obtain final bulk granules, then bagged to obtain sustained-release dry suspension.

### Example 8

Formulation Form

| | |
|---|---|
| Component | Formulation ratio (%) |

50 mg immediate-release pellets

| | | |
|---|---|---|
| Substrate | Microcrystalline pellet | — |
| Immediate-release | Oseltamivir phosphate | 7.88 |
| layer | Hydroxypropyl methyl | 4.00 |
| | cellulose E5 | |
| | Polyethylene glycol | 2.00 |
| | 400 | |
| | Purified water | 86.21 |
| | Total | 100.00 |
| Isolation layer | Opadry 85F | 15.00 |
| | Purified water | 85.00 |
| | Total | 100.00 |

100 mg sustained-release pellets

| | | |
|---|---|---|
| Substrate | Immediate-release pellets | — |
| Sustained-release | Ethyl cellulose | 8.00 |
| coating | Polyethylene glycol 6000 | 4.00 |
| | Triethyl citrate | 2.00 |
| | Purified water | 6.00 |
| | ethanol | 80.00 |
| | Total | 100.00 |
| Isolation layer | Opadry 85F | 15.00 |
| | Purified water | 85.00 |
| | Total | 100.00 |

Preparation of immediate-release pellets: an immediate-release layer coating solution was prepared, and the microcrystalline pellet was used as substrate for coating, after the immediate-release layer coating was completed, a 2% weight-increased isolation layer was wrapped around the pellet to obtain immediate-release pellets;

Preparation of sustained-release pellets: a sustained-release coating solution was prepared, the immediate-release pellets were used as substrate for sustained-release coating, and the percentage of coating weight gain in the immediate-release pellets was 10%, after the sustained-release layer coating was completed, a 2% weight-increased isolation layer was wrapped around the pellet to obtain sustained-release pellets;

Capsule filling: 50 mg of immediate-release pellets and 100 mg of sustained-release pellets were filled into capsules to obtain oseltamivir phosphate biphasic release capsules.

### Example 9

Formulation Form

| | |
|---|---|
| Component | Formulation ratio (%) |

50 mg immediate-release pellets

| | | |
|---|---|---|
| Immediate-release | Oseltamivir phosphate | 36.49 |
| pellet | Microcrystalline cellulose | 51.16 |
| | Pregelatinized starch | 10.00 |
| | Polyvinylpyrrolidone | 2.35 |
| | Total | 100.00 |
| Isolation layer | Opadry 85F | 15.00 |
| | Purified water | 85.00 |
| | Total | 100.00 |

100 mg sustained-release pellets

| | | |
|---|---|---|
| Substrate | Immediate-release pellet | — |
| Sustained-release | Ethyl cellulose | 8.00 |
| coating | Polyethylene glycol 6000 | 4.00 |
| | Triethyl citrate | 2.00 |
| | Purified water | 6.00 |
| | ethanol | 80.00 |
| | Total | 100.00 |
| Isolation layer | Opadry 85F | 15.00 |
| | Purified water | 85.00 |
| | Total | 100.00 |

Preparation of immediate-release pellets: according to the immediate-release pellet formulation, all components (except polyvinylpyrrolidone) were weighed and transferred to a granulator for premixing, and polyvinylpyrrolidone-water was used as granulation solution for wet granulation (the amount of the final dried polyvinylpyrrolidone is shown in the formulation), then wet granulated, extruded and spheronized, dried and screened in turn; the isolation layer coating solution was prepared the sieved pellets were taken and coated, the percentage of coating weight gain in pellets was 2%, and the immediate-release pellets were obtained;

Preparation of sustained-release pellets: according to the sustained-release coating formulation, a coating solution was prepared, and the immediate-release pellets were used as substrate for coating, and the percentage of coating weight gain in the immediate-release pellets was 12%, the sustained-release pellets were obtained. According to the formulation, the isolation layer coating solution was prepared, the sustained-release pellets were taken and coated, the percentage of coating weight gain in the sustained-release pellets was 2%, the final sustained-release pellets were obtained;

Capsule filling: 50 mg of immediate-release pellets and 100 mg of sustained-release pellets were filled into capsules to obtain oseltamivir phosphate biphasic release capsules.

### Example 10

Formulation Form

| | |
|---|---|
| component | Formulation ratio (%) |

75 mg pulsed-release pellets

| | | |
|---|---|---|
| Immediate-release | Oseltamivir phosphate | 43.78 |
| pellets | Microcrystalline cellulose | 28.02 |
| | Mannitol | 10.00 |
| | Sodium chloride | 5.00 |
| | Cross-linked | 10.00 |
| | polyvinylpyrrolidone | |
| | Polyvinylpyrrolidone | 3.20 |
| | Total | 100.00 |
| Sustained-release | Ethyl cellulose | 8.00 |
| layer | Polyethylene glycol 4000 | 4.00 |
| | Triethyl citrate | 1.50 |
| | Purified water | 6.00 |
| | ethanol | 80.50 |
| | Total | 100.00 |
| Isolation layer | Opadry 85F | 15.00 |
| | Purified water | 85.00 |
| | Total | 100.00 |

225 mg delayed sustained-release pellets

| | | |
|---|---|---|
| drug-layered | Oseltamivir phosphate | 49.26 |
| pellet | Microcrystalline | 22.54 |
| | cellulose | |
| | Polyoxyethylene 205 | 10.00 |
| | Sodium chloride | 5.00 |
| | Cross-linked | 10.00 |
| | polyvinylpyrrolidone | |
| | Polyvinylpyrrolidone | 3.20 |
| | Total | 100.00 |
| Sustained-release | Ethyl cellulose | 8.00 |
| layer | Polyethylene glycol | 4.00 |
| | 4000 | |
| | Triethyl citrate | 1.50 |
| | Purified water | 6.00 |
| | ethanol | 80.50 |
| | Total | 100.00 |
| Isolation layer | Opadry 85F | 15.00 |
| | Purified water | 85.00 |
| | Total | 100.00 |

75 mg pulsed-release pellets:

Preparation of immediate-release pellets: according to the formulation of immediate-release pellets, all components (except polyvinylpyrrolidone) were weighed and transferred to a granulator for premixing, and polyvinylpyrrolidone-water was used as granulation solution for wet granulated (the amount of the final dried polyvinylpyrrolidone is shown in the formulation), and then extruded and spheronized, dried and sieved in sequence;

Preparation of pulsed-release pellets: a sustained-release coating solution was prepared, immediate-release pellets were taken and coated, the percentage of coating weight gain in the immediate-release pellets was 8%, and the sustained-release pellets were obtained. the isolation layer coating solution was prepared, the sustained-release pellets were taken and coated, the percentage of coating weight gain in the sustained-release pellets was 2%, and the final pulsed-release pellets were obtained.

225 mg Delayed Sustained-Release Pellets

Preparation of sustained-release pellets: according to the formulation of drug-layered pellet, all components (except polyvinylpyrrolidone) were weighed and transferred to a granulator for premixing, and polyvinylpyrrolidone-water was used as granulation solution for wet granulation (the amount of the final dried polyvinylpyrrolidone is shown in the formulation), then extruded and spheronized, dried and sieved in turn to obtain the drug-layered pellets;

Preparation of pulsed sustained-release pellets: a sustained-release coating solution was prepared, the drug-layered pellets were taken and coated, the percentage of coating weight gain in sustained-release pellets was 10%. the isolation layer coating solution was prepared, the pulsed sustained-release pellets were taken and coated, the percentage of coating weight gain in the pulsed sustained-release pellets was 2%, then the delayed sustained-release pellets were obtained.

Capsule filling: 75 mg of pulsed-release pellets and 225 mg of delayed sustained-release pellets were filled into capsules to obtain oseltamivir phosphate delayed-release capsules.

### Example 11

Formulation Form
150 mg oseltamivir sustained-release formulation

| | Component | Formulation ratio (%) |
|---|---|---|
| Sustained-release | Oseltamivir phosphate | 41.04 |
| layer | Microcrystalline cellulose | 30.00 |
| | 101 QD | |
| | Hydroxypropyl methyl | 15.00 |
| | cellulose K100 LV | |
| | Polyvinylpyrrolidone | 3.00 |
| | Lactose | 4.96 |
| | Micro powder silica | 1.00 |
| | Sodium stearyl fumarate | 5.00 |
| | Total | 100.00 |
| Immediate-release | Oseltamivir phosphate | 43.79 |
| layer | Microcrystalline cellulose | 39.21 |
| | Polyvinylpyrrolidone | 3.00 |
| | Sodium carboxymethyl | 10.00 |
| | starch | |
| | Micro powder silica | 1.00 |
| | Sodium stearyl fumarate | 3.00 |
| | Total | 100.00 |
| Isolation layer | Opadry 85F | 15.00 |
| | Purified water | 85.00 |
| | Total | 100.00 |

Preparation of sustained-release layer bulk granules: according to the formulation of sustained-release layer, oseltamivir phosphate, microcrystalline cellulose and hydroxypropyl methyl cellulose were weighed and transferred to a granulator for premixing, and polyvinylpyrrolidone-water was used as granulation solution for wet granulation (the amount of the final dried polyvinylpyrrolidone is shown in the formulation), then wet granulated, fluidized bed drying, dry granulated in sequence, lactose and lubricant were added to obtain the sustained-release layer bulk granules.

Preparation of immediate-release layer bulk granules: oseltamivir phosphate and microcrystalline cellulose were weighed and transferred to a granulator for premixing, and polyvinylpyrrolidone-water was used as granulation solution for wet granulation (the amount of the final dried polyvinylpyrrolidone is shown in the formulation), then wet granulated, fluidized bed drying, dry granulated in sequence, disintegrant and lubricant were added to obtain the immediate-release layer bulk granules.

Preparation of double-layer tablet: a double-layer tablet press was used to fill 100 mg of sustained-release layer bulk granules, then pre-pressed to obtain the sustained-release layer tablet core; then 50 mg of immediate-release layer granules were filled and pressed into tablet to obtain the immediate-release and sustained-release double-layered tablet core.

Isolation layer: an isolation layer coating solution was prepared and used to coat the above-mentioned immediate-release and sustained-release double-layered tablet core, and the percentage of coating weight gain in the tablet core was 2%.

### Example 12

Formulation Form
150 mg oseltamivir sustained-release formulation

| | Component | Formulation ratio (%) |
|---|---|---|
| Tablet core | Oseltamivir phosphate | 25.60 |
| | Microcrystalline cellulose | 33.35 |
| | 101QD | |
| | Hydroxypropyl methyl | 14.62 |
| | cellulose K100M | |
| | Hydroxypropyl methyl | 19.49 |
| | cellulose K100 LV | |
| | Polyvinylpyrrolidone | 2.56 |
| | Micro powder silica | 0.97 |
| | Sodium stearyl fumarate | 3.41 |
| | Total | 100.00 |
| Controlled- | Hypromellose acetate succinate | 6.00 |
| release film | Triethyl citrate | 0.30 |
| | 80% Ethanol | 93.70 |
| | Total | 100.00 |
| Immediate- | Oseltamivir phosphate | 7.88 |
| release layer | Hydroxypropyl | 4.00 |
| | methylcellulose E5 | |
| | Polyethylene glycol 4000 | 2.00 |
| | Purified water | 86.21 |
| | Total | 100.00 |
| Isolation | Opadry 85F | 15.00 |
| layer | Purified water | 85.00 |
| | Total | 100.00 |

Preparation of tablet core: oseltamivir phosphate, microcrystalline cellulose and hydroxypropyl methyl cellulose were weighed and transferred to a granulator for premixing, and polyvinylpyrrolidone-water was used as granulation solution for wet granulation (the amount of the final dried polyvinylpyrrolidone is shown in the formulation), then wet granulated, fluidized bed drying, dry granulated in sequence, lubricant was added and the mixture was pressed into tablet to obtain a 120 mg oseltamivir sustained-release tablet core.

Controlled-release film: the controlled-release film coating solution was prepared and used to coat the above-mentioned sustained-release tablet core, the percentage of coating weight gain in the tablet core was 2%.

Immediate-release layer: the immediate-release layer coating solution was prepared and used to coat the above-mentioned controlled-release coated tablet, and the coating film contains 30 mg of oseltamivir.

Isolation layer: the isolation layer coating solution was prepared and used to coat the above-mentioned immediate-release coated tablet, and the percentage of coating weight gain in the immediate-release coated tablet was 3%.

### Example 13

Formulation Form
300 mg oseltamivir sustained-release formulation

| | Component | Formulation ratio (%) |
|---|---|---|
| Sustained-release | Oseltamivir phosphate | 67.03 |
| tablet core | Microcrystalline cellulose | 7.02 |
| | Hydroxypropyl methyl | 20.00 |
| | cellulose K100 Lv | |
| | Polyvinylpyrrolidone | 2.45 |
| | Micro powder silica | 1.00 |
| | Magnesium stearate | 2.50 |
| | Total | 100.00 |
| Isolation layer | Opadry 85F | 15.00 |
| | Purified water | 85.00 |
| | Total | 100.00 |

Preparation of tablet core: oseltamivir phosphate, microcrystalline cellulose and hydroxypropyl methyl cellulose were weighed and transferred to a granulator for premixing, polyvinylpyrrolidone-water was used as granulation solution for wet granulation (the amount of the final dried polyvinylpyrrolidone is shown in the formulation), then wet granulated, fluidized bed drying, dry granulated in sequence, lubricant was added to obtain sustained-release layer bulk granules, then pressed into tablet to obtain a 300 mg oseltamivir sustained-release tablet core.

Isolation layer: the isolation layer coating solution was prepared and used to coat the above-mentioned sustained-release tablet core, the percentage of coating weight gain in the tablet core was 2%.

### Example 14

Formulation Form

| | |
|---|---|
| Formulation component | Formulation ratio (%) |

50 mg immediate-release pellets

| | | |
|---|---|---|
| Immediate-release | Oseltamivir phosphate | 43.78 |
| pellets | Microcrystalline cellulose | 28.02 |
| | Mannitol | 10.00 |
| | Sodium chloride | 5.00 |
| | Cross-linked | 10.00 |
| | polyvinylpyrrolidone | |
| | Polyvinylpyrrolidone | 3.20 |
| | Total | 100.00 |
| Isolation layer | Opadry 85F | 15.00 |
| | Purified water | 85.00 |
| | Total | 100.00 |

50 mg pulsed-release pellets

| | | |
|---|---|---|
| | Immediate-release | — |
| | pellets | |
| Sustained-release | Ethyl cellulose | 8.00 |
| layer | Polyethylene glycol | 4.00 |
| | 4000 | |
| | Triethyl citrate | 1.50 |
| | Purified water | 6.00 |
| | ethanol | 80.50 |
| | Total | 100.00 |
| Isolation layer | Opadry 85F | 15.00 |
| | Purified water | 85.00 |
| | Total | 100.00 |

50 mg immediate-release pellets:

Preparation of immediate-release pellets: according to the immediate-release pellets formulation, all components (except polyvinylpyrrolidone) were weighed and transferred to a granulator for premixing, polyvinylpyrrolidone-water was used as granulation solution for wet granulation (the amount of the final dried polyvinylpyrrolidone is shown in the formulation), then extruded and spheronized, dried and screened in turn; the isolation layer coating solution was prepared the sieved pellets were taken and coated, the percentage of coating weight gain in the immediate-release pellets was 2%, and the final immediate-release pellets were obtained.

50 mg pulsed-release pellets:

Preparation of pulsed-release pellets: a sustained-release coating solution was prepared, immediate-release pellets were taken and coated with 2 different weight gain, the percentage of coating weight gain in the pellets was 8% or 30% respectively, and the sustained-release pellets were obtained. the isolation layer coating solution was prepared, the sustained-release pellets with different coating weight gain were taken and coated, the percentage of coating weight gain in the sustained-release pellets was 2%, and the 2-4 h and 7-9 h pulsed-release pellets were obtained.

Capsule filling: 50 mg of immediate-release pellets, 50 mg of pulsed-release pellets (2-4 h release) and 50 mg of pulsed-release pellets (7-9 h release) were filled into capsules to obtain oseltamivir phosphate sustained-release capsules.

### Example 15

Formulation Form
150 mg sustained-release pellets

| | Component | Formulation ratio (%) |
|---|---|---|
| Substrate | Microcrystalline pellet | — |
| Permeation-enhanced | Sodium chloride | 5.00 |
| layer | Hydroxypropyl methyl | 2.00 |
| | cellulose E5 | |
| | Purified water | 93.00 |
| | Total | 100.00 |
| drug layer | Oseltamivir phosphate | 18.00 |
| | Hydroxypropyl methyl | 2.00 |
| | cellulose E5 | |
| | Purified water | 28.00 |
| | 95% Ethanol | 52.00 |
| | Total | 100.00 |
| Sustained-release | Ethyl cellulose E7 | 8.00 |
| layer | Hydroxypropyl cellulose | 0.50 |
| | EF | |
| | Triethyl citrate | 1.50 |
| | 95% Ethanol | 90.00 |
| | Total | 100.00 |
| Isolation layer | Opadry 85F | 15.00 |
| | Purified water | 85.00 |
| | Total | 100.00 |

Preparation of sustained-release pellets: a permeation-enhanced layer coating solution was prepared, the microcrystalline pellet was used as substrate for coating, the percentage of coating weight gain in the pellet was 7%. Then, the drug layer was prepared permeation-enhanced layer pellet was used as substrate for coating, and the percentage of coating weight gain in permeation-enhanced layer pellet was 150%. Then, the sustained-release coating solution was prepared and the upper drug pellet was used as substrate for sustained-release coating, the percentage of coating weight gain in the upper drug pellet was 25%. Then, the isolation layer coating solution was prepared and an isolation layer was wrapped around the sustained-release layer, the percentage of coating weight gain in sustained-release pellet was 2%. Finally, 150 mg of oseltamivir sustained-release pellets were filled into capsules to obtain oseltamivir phosphate sustained-release capsules.

### Example 16

Formulation Form

| | |
|---|---|
| Component | Formulation ratio (%) |

30 mg immediate-release pellets

| | | |
|---|---|---|
| Substrate | Microcrystalline pellet | — |
| drug layer | Oseltamivir phosphate | 18.00 |
| | Hydroxypropyl methyl | 2.00 |
| | cellulose E5 | |
| | Purified water | 28.00 |
| | 95% Ethanol | 52.00 |
| | Total | 100.00 |
| Isolation layer | Opadry 85F | 15.00 |
| | Purified water | 85.00 |
| | Total | 100.00 |

120 mg sustained-release pellets

| | | |
|---|---|---|
| Substrate | Microcrystalline pellet | — |
| Permeation-enhanced | Sodium chloride | 5.00 |
| layer | Hydroxypropyl methyl | 2.00 |
| | cellulose E5 | |
| | Purified water | 93.00 |
| | Total | 100.00 |
| Drug layer | Oseltamivir phosphate | 18.00 |
| | Hydroxypropyl methyl | 2.00 |
| | cellulose E5 | |
| | Purified water | 28.00 |
| | 95% Ethanol | 52.00 |
| | Total | 100.00 |
| Sustained-release | Ethyl cellulose E7 | 8.00 |
| layer | Hydroxypropyl cellulose | 0.50 |
| | EF | |
| | Triethyl citrate | 1.50 |
| | 95% Ethanol | 90.00 |
| | Total | 100.00 |
| Isolation layer | Opadry 85F | 15.00 |
| | Purified water | 85.00 |
| | Total | 100.00 |

Preparation of immediate-release pellets: a drug layer coating solution was prepared according to the formulation of immediate-release pellets, and the microcrystalline pellet was used as substrate for coating, the percentage of coating weight gain in the pellet was 150%; after the coating was completed, a 2% weight-increasing isolation layer was wrapped around the drug layer to obtain immediate-release pellets;

Preparation of sustained-release pellets: according to the formulation of sustained-release pellets, the permeation-enhanced layer coating solution was prepared, and the microcrystalline pellet was used as substrate for coating, the percentage of coating weight gain in the pellet was 7%. Then, the drug layer was prepared permeation-enhanced layer pellet was used as substrate for coating, and the percentage of coating weight gain in permeation-enhanced layer pellet was 150%. Then, the sustained-release coating solution was prepared and the upper drug pellet was used as substrate for sustained-release coating, the percentage of coating weight gain in the upper drug pellet was 25%. Then, the isolation layer coating solution was prepared and the sustained-release layer was coated with an isolation layer, the percentage of coating weight gain in the sustained-release pellet was 2%, and the sustained-release pellets were obtained.

Capsule filling: 30 mg of immediate-release pellets and 120 mg of sustained-release pellets were filled into capsules to obtain oseltamivir phosphate biphasic release capsules.

### Example 17

Formulation Form

| | |
|---|---|
| Component | Formulation ratio (%) |

75 mg immediate-release pellets

| | | |
|---|---|---|
| Substrate | Microcrystalline pellet | — |
| Drug layer | Oseltamivir phosphate | 18.00 |
| | Hydroxypropyl methyl | 2.00 |
| | cellulose E5 | |
| | Purified water | 28.00 |
| | 95% Ethanol | 52.00 |
| | Total | 100.00 |
| Isolation layer | Opadry 85F | 15.00 |
| | Purified water | 85.00 |
| | Total | 100.00 |

75 mg sustained-release pellets

| | | |
|---|---|---|
| Substrate | Microcrystalline pellet | — |
| Permeation-enhanced | Sodium chloride | 5.00 |
| layer | Hydroxypropyl methyl | 2.00 |
| | cellulose E5 | |
| | Purified water | 93.00 |
| | Total | 100.00 |
| Drug layer | Oseltamivir phosphate | 18.00 |
| | Hydroxypropyl methyl | 2.00 |
| | cellulose E5 | |
| | Purified water | 28.00 |
| | 95% Ethanol | 52.00 |
| | Total | 100.00 |
| Sustained-release | Ethyl cellulose E7 | 8.00 |
| layer | Hydroxypropyl cellulose | 0.50 |
| | EF | |
| | Triethyl citrate | 1.50 |
| | 95% Ethanol | 90.00 |
| | Total | 100.00 |
| Isolation layer | Opadry 85F | 15.00 |
| | Purified water | 85.00 |
| | Total | 100.00 |

Preparation of immediate-release pellets: a drug layer coating solution was prepared according to the formulation of immediate-release pellets, and the microcrystalline pellet was used as substrate for coating, the percentage of coating weight gain in the pellet was 150%; after the coating was completed, a 2% weight-increasing isolation layer was wrapped around the drug layer to obtain immediate-release pellets;

Preparation of sustained-release pellets: according to the formulation of sustained-release pellets, the permeation-enhanced layer coating solution was prepared, and the microcrystalline pellet was used as substrate for coating, the percentage of coating weight gain in the pellet was 7%. Then, the drug layer was prepared permeation-enhanced layer pellet was used as substrate for coating, and the percentage of coating weight gain in permeation-enhanced layer pellet was 150%. Then, the sustained-release coating solution was prepared and the upper drug pellet was used as substrate for sustained-release coating, the percentage of coating weight gain in the upper drug pellet was 30%. Then, the isolation layer coating solution was prepared and the sustained-release layer was coated with an isolation layer, the percentage of coating weight gain in the sustained-release pellet was 2%, and the sustained-release pellets were obtained.

Capsule filling: 75 mg of immediate-release pellets and 75 mg of sustained-release pellets were filled into capsules to obtain oseltamivir phosphate biphasic release capsules.

### Comparative Example 1: Weight Ratio of Oseltamivir was 2%, Content Uniformity was Poor (Compared with Example 7)

Formulation Form
10 mg immediate-release granules

| component | Formulation ratio (%) |
|---|---|
| Oseltamivir phosphate | 8.54 |
| Sucrose | 90.59 |
| PVP K29/32 | 0.87 |
| Total | 100.00 |

50 mg sustained-release granules

| | component | Formulation ratio (%) |
|---|---|---|
| Immediate-release | Oseltamivir phosphate | 8.54 |
| granules | Microcrystalline cellulose | 90.59 |
| | Polyvinylpyrrolidone | 0.87 |
| | Total | 100.00 |
| Sustained-release | Ethyl cellulose | 8.00 |
| coating layer | Polyethylene glycol 6000 | 4.00 |
| | Triethyl citrate | 2.00 |
| | Purified water | 6.00 |
| | ethanol | 80.00 |
| | Total | 100.00 |

| | |
|---|---|
| component | Formulation ratio (%) |

Blank granules

| | |
|---|---|
| Sucrose | 97.41 |
| Xanthan Gum | 1.00 |
| Sodium citrate dihydrate | 0.50 |
| Sodium benzoate | 0.25 |
| Polyvinylpyrrolidone | 0.84 |
| Total | 100.00 |

60 mg oseltamivir sustained-release formulation

| | |
|---|---|
| 10 mg immediate-release granules | 2.17 |
| 50 mg sustained-release granules | 14.63 |
| Blank granules | 79.46 |
| Sucralose | 2.00 |
| Orange essence | 0.75 |
| Micro powder silica | 1.00 |
| Total | 100.00 |

Preparation of immediate-release granules: oseltamivir phosphate and sucrose were weighed and transferred to a granulator for premixing, and polyvinylpyrrolidone-water was used as granulation solution for wet granulation (the amount of the final dried polyvinylpyrrolidone is shown in the formulation), and then wet granulated, fluidized bed drying, and dry granulated in sequence to obtain the immediate-release granules.

Preparation of sustained-release granules: sustained-release coating solution was prepared, then immediate-release granules were taken and sustained-release coated, the percentage of coating weight gain in the granules was 35%.

Preparation of blank granules: according to the formulation of blank granules, all components (except polyvinylpyrrolidone) were weighed and transferred to a granulator for premixing, polyvinylpyrrolidone-water was used as granulation solution for wet granulation (the amount of the final dried polyvinylpyrrolidone is shown in the formulation), and then wet granulated, fluidized bed drying, and dry granulated in turn to obtain the blank granules.

Preparation of bulk granules: 10 mg of immediate-release granules, 50 mg of sustained-release granules, blank granules were weighed, then flavoring agent and glidant were added and mixed to obtain the final bulk granules.

Experimental results: the content uniformity of the bulk granules in Comparative Example 1 (2% oseltamivir content) is poor, and the content uniformity of the bulk granules in Example 7 (3% oseltamivir content) is qualified.

TABLE 1
Content uniformity of the bulk granules
in Comparative Example 1 and Example 7
Content uniformity of the bulk granules (%)

| Sampling location | Comparative Example 1 | Example 7 |
|---|---|---|
| T1 | 110.4 | 100.1 |
| T2 | 105.4 | 99.8 |
| T3 | 101.9 | 100.7 |
| M1 | 94.3 | 99.5 |
| M2 | 105.5 | 99.8 |
| M3 | 92.7 | 98.9 |
| B1 | 85.3 | 101.7 |
| RSD(%) | 8.24% | 0.83% |

Formulation Form
300 mg oseltamivir sustained-release formulation

| Component | Formulation ratio (%) |
|---|---|
| Oseltamivir phosphate | 72.31 |
| Microcrystalline cellulose | 2.19 |
| Hydroxypropyl methyl cellulose K100LV | 20.00 |
| Polyvinylpyrrolidone | 1.50 |
| Micro powder silica | 1.00 |
| Magnesium stearate | 3.00 |
| Total | 100.00 |

Preparation of tablet cores: oseltamivir phosphate, microcrystalline cellulose and hydroxypropyl methylcellulose were weighed and transferred to a granulator for premixing, and polyvinylpyrrolidone-water was used as granulation solution for wet granulation (the amount of the final dried polyvinylpyrrolidone is shown in the formulation), then wet granulated, fluidized bed drying, dry granulated in sequence, and lubricant was added to obtain the sustained-release layer bulk granules, then compressed into tablets.

Experimental results: the tableting process showed serious sticking and punching, and the surface of the tablet was uneven.

Formulation Form
180 mg oseltamivir sustained-release formulation

| | Component | Formulation ratio (%) |
|---|---|---|
| Tablet core | Oseltamivir phosphate | 41.15 |
| | Mannitol | 29.52 |
| | Polyvinylpyrrolidone | 1.23 |
| | Microcrystalline cellulose | 16.60 |
| | Sodium chloride | 7.50 |
| | Micro powder silica | 1.00 |
| | Sodium stearate fumarate | 3.00 |
| | Total | 100.00 |
| Controlled-release | Ethyl cellulose | 8.00 |
| film | Polyethylene glycol 4000 | 4.00 |
| | Triethyl citrate | 2.00 |
| | Ethanol | 86.00 |
| | Total | 100.00 |
| Immediate-release | Oseltamivir phosphate | 7.88 |
| layer | Hydroxypropyl methyl | 4.00 |
| | cellulose E5 | |
| | Polyethylene glycol 400 | 2.00 |
| | Purified water | 86.21 |
| | Total | 100.00 |
| Isolation layer | Opadry 85F | 15.00 |
| | Purified water | 85.00 |
| | Total | 100.00 |

Preparation of tablet cores: oseltamivir phosphate and mannitol were weighed and transferred to a granulator for premixing, polyvinylpyrrolidone-water was used as granulation solution for wet granulation (the amount of the final dried polyvinylpyrrolidone is shown in the formulation), then wet granulated, fluidized bed drying, and dry granulated in turn, and filler, penetration enhancer and lubricant were added and the mixture was pressed into tablets to obtain 130 mg of oseltamivir tablet cores.

Controlled-release film: the controlled-release coating solution was prepared and used to coat the tablet core, the percentage of coating weight gain in the tablet core was 6%.

Immediate-release layer: the immediate-release layer coating solution was prepared and used to coat the above-mentioned controlled-release coated tablet, and the coating film contains 50 mg of oseltamivir.

Isolation layer: the isolation layer coating solution was prepared and used to coat the above-mentioned immediate-release coated tablet, and the percentage of coating weight gain in the immediate-release coated tablet was 2%.

TABLE 2
Dissolution data of Comparative Example 3 and Example 3
Ph 6.8 medium dissolution (%)

| | Comparative Example 3 | Example 3 |
|---|---|---|
| Time(h) | (Mean + SD) | (Mean + SD) |
| 0.25 | 21 ± 15.04 | 20 ± 1.02 |
| 1 | 44 ± 10.05 | 42 ± 0.80 |
| 2 | 58 ± 12.03 | 56 ± 0.58 |
| 3 | 69 ± 15.67 | 67 ± 1.04 |
| 4 | 77 ± 8.87 | 75 ± 2.04 |
| 6 | 86 ± 7.78 | 85 ± 1.04 |
| 8 | 94 ± 8.45 | 92 ± 0.68 |
| 10 | 96 ± 2.32 | 96 ± 0.78 |
| 12 | 97 ± 1.12 | 98 ± 0.86 |

Formulation Form
90 mg oseltamivir sustained-release formulation

| | Component | Formulation ratio (%) |
|---|---|---|
| Tablet core | Oseltamivir phosphate | 19.71 |
| | Microcrystalline cellulose | 5.33 |
| | Hydroxypropyl methyl | 70.00 |
| | cellulose K100 LV | |
| | Polyvinylpyrrolidone | 0.97 |
| | Micro powder silica | 1.00 |
| | Sodium stearyl fumarate | 3.00 |
| | Total | 100.00 |
| Immediate-release | Oseltamivir phosphate | 7.88 |
| layer | Hydroxypropyl | 4.00 |
| | methylcellulose E5 | |
| | Polyethylene glycol 400 | 2.00 |
| | Purified water | 86.21 |
| | Total | 100.00 |
| Isolation layer | Opadry 85F | 15.00 |
| | Purified water | 85.00 |
| | Total | 100.00 |

Preparation of tablet cores: oseltamivir phosphate, microcrystalline cellulose and hydroxypropyl methylcellulose were weighed and transferred to a granulator for premixing, polyvinylpyrrolidone-water was used as granulation solution for wet granulation (the amount of the final dried polyvinylpyrrolidone is shown in the formulation), and then wet granulated, fluidized bed drying, dry granulated in sequence, then lubricant was added and the mixture was pressed into tablets to obtain 60 mg of oseltamivir sustained-release tablet cores.

Immediate-release layer: the immediate-release layer coating solution was prepared and used to coat the above-mentioned sustained-release tablet core, and the coating film contains 30 mg of oseltamivir.

Isolation layer: the isolation layer coating solution was prepared and used to coat the above-mentioned immediate-release coated tablet, and the percentage of coating weight gain in the immediate-release coated tablet was 2%.

Experimental results: Comparative example 4 has a slow dissolution, and the dissolution at 10 h is less than 70%.

TABLE 3
Dissolution data of Comparative Example 4 and Example 4
Ph 6.8 medium dissolution (%)

| | Comparative Example 4 | Example 4 |
|---|---|---|
| Time(h) | (mean) | (mean) |
| 0.25 | 29 | 28 |
| 1 | 36 | 37 |
| 2 | 42 | 44 |
| 3 | 48 | 50 |
| 4 | 53 | 56 |
| 6 | 60 | 64 |
| 8 | 65 | 72 |
| 10 | 69 | 77 |
| 12 | 73 | 81 |

### Comparative Example 5

Formulation Form
150 mg oseltamivir sustained-release formulation

| | Component | Formulation ratio (%) |
|---|---|---|
| Sustained- | Oseltamivir phosphate | 41.05 |
| release layer | Microcrystalline cellulose 101 QD | 34.99 |
| | Hydroxypropyl methyl cellulose | 10.00 |
| | K100 LV | |
| | Polyvinylpyrrolidone | 3.00 |
| | Lactose | 4.96 |
| | Micro powder silica | 1.00 |
| | Sodium stearyl fumarate | 5.00 |
| | Total | 100.00 |
| Immediate- | Oseltamivir phosphate | 43.79 |
| release layer | Microcrystalline cellulose | 39.21 |
| | Polyvinylpyrrolidone | 3.00 |
| | Sodium carboxymethyl starch | 10.00 |
| | Micro powder silica | 1.00 |
| | Sodium stearyl fumarate | 3.00 |
| | Total | 100.00 |
| Isolation | Opadry 85F | 15.00 |
| layer | Purified water | 85.00 |
| | Total | 100.00 |

Preparation of sustained-release layer bulk granules: oseltamivir phosphate, microcrystalline cellulose and hydroxypropyl methyl cellulose were weighed and transferred to a granulator for pre-mixing, polyvinylpyrrolidone-water was used as granulation solution for wet granulation (the amount of the final dried polyvinylpyrrolidone is shown in the formulation), then wet granulated, fluidized bed drying, dry granulated in turn, lactose and lubricant were added to obtain sustained-release layer bulk granules.

Preparation of immediate-release layer bulk granules: oseltamivir phosphate and microcrystalline cellulose were weighed and transferred to a granulator for premixing, polyvinylpyrrolidone-water was used as granulation solution for wet granulation (the amount of the final dried polyvinylpyrrolidone is shown in the formulation), and then wet granulated, fluidized bed drying, dry granulated, disintegrant and lubricant were added to obtain immediate-release layer bulk granules.

Preparation of double-layer tablet: a double-layer tablet press was used to fill 90 mg of sustained-release layer bulk granules, then pre-pressed to obtain the sustained-release layer tablet core; then 60 mg of immediate-release layer granules were filled and pressed into tablet to obtain the immediate-release and sustained-release double-layered tablet core.

Isolation layer: an isolation layer coating solution was prepared and used to coat the above-mentioned immediate-release and sustained-release double-layered tablet core, and the percentage of coating weight gain in the tablet core was 2%.

### Comparative Example 6

Formulation Form
150 mg oseltamivir sustained-release formulation

| | Component | Formulation ratio (%) |
|---|---|---|
| Tablet core | Oseltamivir phosphate | 40.22 |
| | Microcrystalline cellulose 101QD | 33.58 |
| | Polyvinylpyrrolidone | 2.20 |
| | Lactose | 15.00 |
| | Sodium chloride | 5.00 |
| | Micro powder silica A200 | 1.00 |
| | Sodium stearate fumarate | 3.00 |
| | Total | 100.00 |
| Controlled- | Cellulose acetate | 6.00 |
| release film | PEG 6000 | 1.50 |
| | Purified water | 6.00 |
| | acetone | 86.50 |
| | Total | 100.00 |

Preparation of tablet core: oseltamivir phosphate and microcrystalline cellulose were weighed and transferred to a granulator for premixing, polyvinylpyrrolidone-water was used as granulation solution for wet granulation (the amount of the final dried polyvinylpyrrolidone is shown in the formulation), then wet granulated, fluidized bed drying, dry granulated in turn, then filler, penetration enhancer and lubricant were added, and the mixture was pressed into tablets to obtain a 150 mg of oseltamivir tablet core.

Controlled-release film: the controlled-release coating solution was prepared and used to coat the above-mentioned tablet core, the percentage of coating weight gain in the tablet core was 12%.

Dissolution Data

In this example, the oseltamivir formulations prepared in Examples 1 to 14 were dissolved in a pH 6.8 medium, the volume of the medium was 900±9 mL, the temperature of the medium was 37.0±0.5° C., the paddle method was used, and the in vitro dissolution test was performed at 50 rpm/min. Time points for dissolution sampling in pH6.8: 0.25 h, 1 h, 2 h, 3 h, 4 h, 6 h, 8 h, 10 h, 12 h. Sampling location: from the top of the paddle to the midpoint of the liquid surface, 10 mm away from the inner wall of the dissolution vessel. In vitro dissolution content was tested by High Performance Liquid Chromatography (HPLC) method. Results are as shown below:

TABLE 4
Dissolution (%) of the products of each
example in pH 6.8 medium (n = 3)

| Test number | 0.25 h | 1 h | 2 h | 3 h | 4 h | 6 h | 8 h | 10 h | 12 h |
|---|---|---|---|---|---|---|---|---|---|
| Example 1 | 20 | 45 | 60 | 70 | 78 | 89 | 95 | 97 | 99 |
| Example 2 | 32 | 48 | 61 | 71 | 78 | 87 | 94 | 97 | 98 |
| Example 3 | 20 | 42 | 56 | 67 | 75 | 85 | 92 | 96 | 98 |
| Example 4 | 28 | 37 | 44 | 50 | 56 | 64 | 72 | 77 | 81 |
| Example 5 | 31 | 45 | 52 | 58 | 63 | 72 | 80 | 86 | 92 |
| Example 6 | 27 | 40 | 49 | 56 | 62 | 73 | 84 | 95 | 97 |
| Example 7 | 16 | 31 | 45 | 58 | 68 | 83 | 92 | 97 | 98 |
| Example 8 | 34 | 45 | — | — | 65 | 81 | — | 95 | — |
| Example 9 | 33 | 46 | — | — | 69 | 84 | — | 93 | 97 |
| Example 10 | 0 | 0 | 0 | 3 | 25 | 57 | 85 | 94 | 95 |
| Example 11 | 38 | 55 | 72 | 86 | 90 | — | — | 98 | 98 |
| Example 12 | 20 | 35 | 42 | 48 | 53 | 61 | 68 | 74 | 79 |
| Example 13 | 12 | 25 | 36 | 45 | 53 | 66 | 79 | 89 | 94 |
| Example 14 | 25 | 32 | — | — | 65 | — | 84 | 96 | — |
| Example 15 | 0 | 32 | 61 | 74 | 81 | 88 | 92 | 96 | 97 |
| Example 16 | 22 | 45 | 69 | 78 | 83 | 90 | 93 | 96 | 98 |
| Example 17 | 38 | 54 | 73 | 83 | 87 | 91 | 94 | 96 | 97 |
| Comparative | 29 | 36 | 42 | 48 | 53 | 60 | 65 | 69 | 73 |
| Example 4 | | | | | | | | | |
| Comparative | 40 | 61 | 87 | 95 | 98 | — | — | 99 | — |
| Example 5 | | | | | | | | | |
| Comparative | 2 | 8 | 15 | 23 | 31 | 45 | 58 | 69 | 78 |
| Example 6 | | | | | | | | | |
| Immediate-release | 91 | 100 | — | — | — | — | — | — | — |
| formulation | | | | | | | | | |
| (Tamifu ®) | | | | | | | | | |

Table 5

TABLE 5
Summary of the characteristics of the products
of each example and comparative example

| | | | | Release | |
|---|---|---|---|---|---|
| | | | Sustained- | amount for a | Release |
| | | Osel- | release | certain 1 h | amount |
| | Strength | tamivir | material | within <4 | within 4 |
| Test number | (mg) | ratio % | ratio % | h % | h % |
| Example 1 | 150 | 31.6 | 4.4 | 45 | 78 |
| Example 2 | 60 | 22.4 | 3.3 | 45 | 78 |
| Example 3 | 180 | 3.3 | 3.3 | 42 | 75 |
| Example 4 | 90 | 26.7 | 50 | 37 | 56 |
| Example 5 | 150 | 32 | 29 | 45 | 63 |
| Example 6 | 200 | 28 | 14 | 40 | 62 |
| Example 7 | 60 | 3 | 3.3 | 31 | 68 |
| Example 8 | 150 | 8.5 | 3.6 | 45 | 65 |
| Example 9 | 150 | 24.9 | 4.2 | 46 | 69 |
| Example 10 | 300 | 31.6 | 7.6 | 25 | 25 |
| Example 11 | 150 | 31.2 | 10 | 55 | 90 |
| Example 12 | 150 | 21 | 31.2 | 40 | 62 |
| Example 13 | 300 | 50 | 19.6 | 25 | 53 |
| Example 14 | 150 | 29 | 6.5 | 25 | 65 |
| Example 15 | 150 | 32 | 16 | 32 | 81 |
| Example 16 | 150 | 34 | 13 | 45 | 83 |
| Example 17 | 150 | 34 | 11 | 54 | 87 |
| Example 18 | 180 | 30.2 | 37.0 | 33 | 48 |
| Example 19 | 180 | 30.2 | 28.8 | 35 | 57 |
| Example 20 | 180 | 30.2 | 24.7 | 34 | 50 |
| Example 21 | 180 | 25.1 | 50.1 | 31 | 46 |
| Example 22 | 180 | 30.2 | 20.6 | 35 | 54 |
| Example 23 | 180 | 30.2 | 37.0 | 34 | 51 |
| Example 24 | 180 | 30.2 | 28.8 | 32 | 46 |
| Example 25 | 180 | 30.2 | 37.0 | 34 | 51 |
| Example 26 | 180 | 27.6 | 33.8 | 33 | 47 |
| Comparative | 60 | 2 | 2.1 | — | — |
| Example 1 | | | | | |
| Comparative | 300 | 55 | 20 | — | — |
| Example 2 | | | | | |
| Comparative | 180 | 3.3 | 2.5 | 44 | 77 |
| Example 3 | | | | | |
| Comparative | 90 | 18.7 | 55 | 36 | 53 |
| Example 4 | | | | | |
| Comparative | 150 | 31.5 | 6.0 | 61 | 98 |
| Example 5 | | | | | |
| Comparative | 150 | 27.3 | 8.6 | 8 | 31 |
| Example 6 | | | | | |

Remarks: “release amount for a certain 1 h within <4 h” and “release amount within 4 h” in Examples 18 to 26 are the dissolution data of acid resistance in 0.1M HCl for 1 h and then transferred to pH 6.8 medium. Other examples or comparative examples in the table are dissolution data in Ph 6.8 medium.

Animal Test: Pharmacokinetic Study of Oseltamivir Formulation in Beagle Dogs

The First Pharmacokinetic Study of Oseltamivir Formulation in Beagle Dogs

Dosing scheme: the test was used four-formulation, four-period and four-crossover (24 healthy beagle dogs, half male and half male, divided into 4 groups, 6 dogs in each group, administered on an empty stomach). Three groups were administered self-developed oseltamivir sustained-release formulation (150 mg) with once a day; the other groups were administered reference formulation, which was a 75 mg immediate-release formulation (trade name: Tamifu®), twice a day.

Table 6 shows the pharmacokinetic data of the products in Example 11, Comparative Example 5, Comparative Example 6 and the reference formulation in beagle dogs (the concentration of the oseltamivir active metabolite was detected).

Comparative example 5 has a fast release rate and fast absorption, which leads to a much higher Cmax in beagle dogs than the reference formulation, and the risk of toxic and side effects increases; in addition, due to short-term centralized absorption and fast elimination, it cannot maintain a long-term stable effective blood concentration; Comparative Example 6 has a slow release rate and slow absorption, the Cmax in beagle dogs is much lower than that of the reference formulation, which may not have a rapid onset effect, and the bioavailability is only 47% of that of the reference formulation twice a day; and the optimal absorption site may have been missed; the release rate of Example 12 is moderate, the Cmax in beagle dogs is 80% of the reference formulation twice a day, and the bioavailability is 81% of the reference formulation twice a day, which is relatively ideal. Therefore, the product of Example 12 is selected for the first human clinical trial.

TABLE 6
Pharmacokinetic data of the formulations in Example
12, Comparative Example 5, Comparative Example
6 and the reference formulation in beagle dogs

| | | | BA(%) |
|---|---|---|---|
| | | | (Self-developed |
| | Cmax | AUC0-t | formulation/ |
| | ((ng/mL)) | (ng · h/mL) | reference formulation) |
| Example 12 | 3400 ± 780 | 43300 ± 7500 | 91 |
| Comparative | 6790 ± 940 | 45700 ± 6200 | 96 |
| Example 5 | | | |
| Comparative | 2340 ± 670 | 22400 ± 6500 | 47 |
| Example 6 | | | |
| Reference | 4230 ± 760 | 47600 ± 7500 | — |
| formulation | | | |

The Second Pharmacokinetic Study of Oseltamivir Formulation in Beagle Dogs

Dosing scheme: the test was used double-formulation and double-crossover (12 healthy beagle dogs, half male and female, divided into 2 groups, 6 dogs in each group, administered on an empty stomach), both groups were administered self-developed oseltamivir sustained-release formulation (150 mg) with once a day.

Table 7 shows the pharmacokinetic data of the products in Example 5 and Example 12 in beagle dogs (the concentration of the oseltamivir active metabolite was detected).

The AUC₀₋ₜ of the products in Example 5 and Example 12 in beagle dogs is similar, but the Cmax of the product of Example 5 is much higher than that of Example 12. Considering that the high blood concentration, the risk of toxic and side effects may increase. The product of Example 12 was selected for the second human clinical trial.

TABLE 7
Pharmacokinetic data of the formulations in
Example 5 and Example 12 in beagle dogs

| | Cmax ((ng/mL)) | AUC₀₋ₜ (ng · h/mL) |
|---|---|---|
| Example 5 | 4810 ± 750 | 53200 ± 7100 |
| Example 12 | 3980 ± 670 | 48800 ± 6800 |

The Third Pharmacokinetic Study of Oseltamivir Formulation in Beagle Dogs

Dosing scheme: the test was used double-formulation and double-crossover (12 healthy beagle dogs, half male and female, divided into 2 groups, 6 dogs in each group, administered on an empty stomach), both groups were administered self-developed oseltamivir sustained-release formulation (150 mg) with once a day.

Table 8 shows the pharmacokinetic data of the products in Example 18 and Example 5 in beagle dogs (the concentration of the oseltamivir active metabolite was detected).

The AUC₀₋ₜ of the product in Example 18 in beagle dogs was 1.17 times that of Example 5, and the Cmax of the product in Example 18 is slightly higher than that of Example 5. Therefore, the product of Example 18 is selected for the third human clinical trial.

TABLE 8
Pharmacokinetic data of the formulations in
Example 18 and Example 5 in beagle dogs

| | Cmax ((ng/mL)) | AUC0-t (ng · h/mL) |
|---|---|---|
| Example 18 | 3970 ± 610 | 43400 ± 5800 |
| Example 5 | 3580 ± 580 | 37100 ± 5400 |

Clinical Trial: Pharmacokinetic Study of Oseltamivir Formulation in Humans

The First Human Clinical Trial

Dosing regimen: the biphasic release formulation of oseltamivir (150 mg) prepared in Example 12 was administered in an empty stomach once a day.

Table 9 shows the pharmacokinetic data of the biphasic release formulation of oseltamivir prepared in Example 12 in humans (the concentration of the oseltamivir active metabolite was detected). The product of the present invention has good pharmacokinetic properties, the Tmax is 6 h, the Tmax of food administration is 14 h, and the Cmax is greater than 300 ng/mL. The drug-time curve is shown in FIG. 1.

It can be seen from FIG. 1 that after oral administration of the self-developed product, the oseltamivir active metabolite in vivo reaches 150 ng/mL within 3 h to 4 h, which plays a role of rapid onset; the maintenance time of the plasma concentration higher than 150 ng/ml within 24 h is 21 h, and the peak-to-valley concentration ratio of plasma concentration within 24 h is 2.14, which can maintain the effective plasma concentration stably for a long time.

TABLE 9
Pharmacokinetic data of the biphasic release formulation
of oseltamivir prepared in Example 12 in humans

| | Example 12 |
|---|---|
| Tmax(h) | 6 |
| Cmax(ng/mL) | 370 |
| AUC0-t(ng · h/mL) | 8343 |

The Second Human Clinical Trial

Dosing scheme: the test was used double-formulation and double-crossover, one group was administered the biphasic release formulation (150 mg) of oseltamivir prepared in Example 12 in a fed state once a day; the other group was administered reference formulation, which was a 75 mg immediate-release formulation (trade name: Tamifu®), administered twice a day.

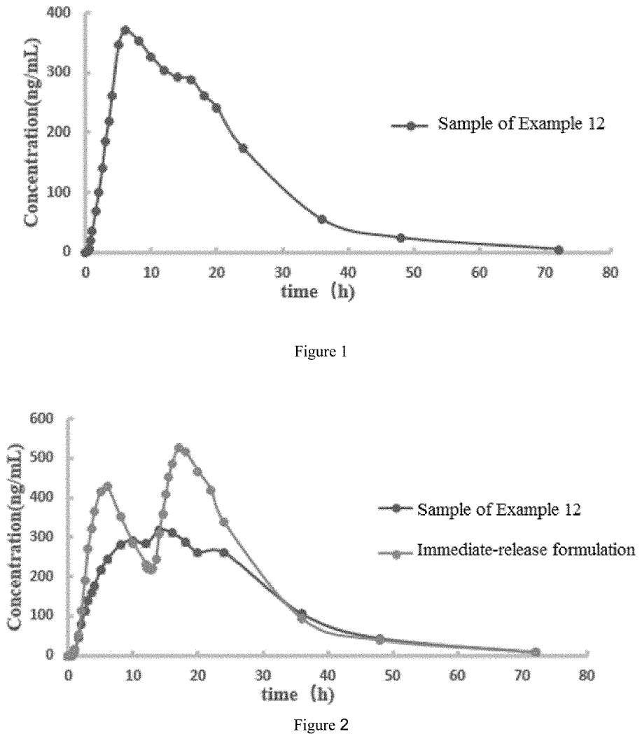

Table 10 shows the pharmacokinetic data of Example 12 and the reference formulation in humans (the concentration of the oseltamivir active metabolite was detected). The product of the present invention has good pharmacokinetic properties, Tmax is 14 h, and Cmax is greater than 300 ng/mL. The drug-time curve is shown in FIG. 2.

It can be seen from FIG. 2 that after oral administration of the self-developed product, the oseltamivir active metabolite in vivo reaches 150 ng/mL within 3 h to 4 h, which plays a role of rapid onset; the maintenance time of the plasma concentration higher than 150 ng/ml within 24 h is 21 h, and the peak-to-valley concentration ratio of plasma concentration within 24 h is 1.21, which can maintain the effective plasma concentration stably for a long time.

TABLE 10
Pharmacokinetic data of the biphasic release formulation
of oseltamivir prepared in Example 12 in humans

| | Example 12 | Reference formulation |
|---|---|---|
| Tmax(h) | 14 | 17 |
| Cmax(ng/mL) | 318 | 526 |
| AUC0-t(ng · h/mL) | 9537 | 12180 |

The Third Human Clinical Trial

Dosing scheme: the test used double-formulation and double-crossover, one group was administered the oseltamivir biphasic release formulation (180 mg) prepared in Example 18 in the state of eating once a day; the other group was administered reference formulation, which was a 75 mg immediate-release formulation (trade name: Tamifu®), administered twice a day.

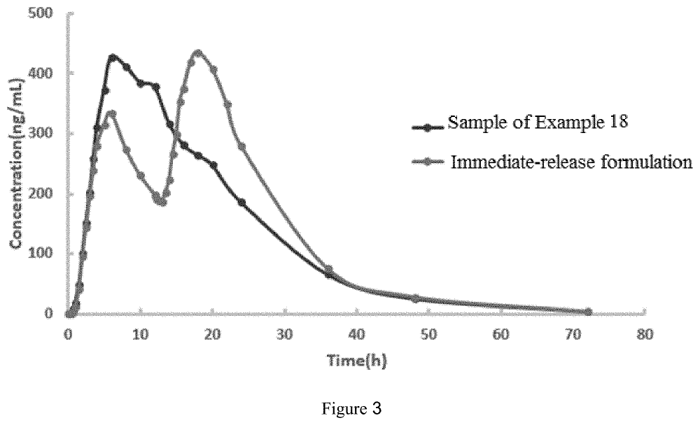

Table 11 shows the pharmacokinetic data of Example 18 and the reference formulation in humans (the concentration of the oseltamivir active metabolite was detected). The product of the present invention has good pharmacokinetic properties, Tmax is 8 h, and Cmax is greater than 300 ng/mL. The drug-time curve is shown in FIG. 3.

It can be seen from FIG. 3 that after oral administration of the self-developed product, the oseltamivir active metabolite in vivo reaches 150 ng/mL within 2 h to 3 h, which is equivalent to the reference formulation and has a rapid onset effect; the maintenance time of the plasma concentration higher than 150 ng/ml within 24 h is 21.5 h, and the peak-to-valley concentration ratio of plasma concentration within 24 h is 2.30, which can maintain the effective plasma concentration stably for a long time.

TABLE 11
Pharmacokinetic data of the biphasic release formulation
of oseltamivir prepared in Example 18 in humans

| | Example 18 | Reference formulation |
|---|---|---|
| Tmax(h) | 8 | 16 |
| Cmax(ng/mL) | 455 | 463 |
| AUC0-t(ng · h/mL) | 9206 | 9737 |

### Example 18

Formulation Form
180 mg oseltamivir sustained-release formulation

| | Component | Formulation ratio (%) |
|---|---|---|
| Sustained-release | Oseltamivir phosphate | 40.22 |
| layer | MCC 101QD | 9.78 |
| | Methacrylic acid-ethyl | 30.00 |
| | acrylate copolymer | |
| | L100-55 | |
| | Hydroxypropyl methyl | 15.00 |
| | cellulose K15M | |
| | Sodium stearate fumarate | 5.00 |
| | Total | 100.00 |
| Immediate-release | Oseltamivir phosphate | 41.93 |
| layer | MCC 101QD | 44.00 |
| | Polyvinylpyrrolidone | 1.07 |
| | Croscarmellose sodium | 10.00 |
| | Sodium stearate fumarate | 3.00 |
| | Total | 100.00 |
| Isolation layer | Opadry 85F | 15.00 |
| | Purified water | 85.00 |
| | Total | 100.00 |

### Example 19

Formulation Form
180 mg oseltamivir sustained-release formulation

| | Component | Formulation ratio (%) |
|---|---|---|
| Sustained-release | Oseltamivir phosphate | 40.22 |
| layer | MCC 101QD | 19.78 |
| | Methacrylic acid-ethyl | 30.00 |
| | acrylate copolymer | |
| | L100-55 | |
| | Hydroxypropyl methyl | 5.00 |
| | cellulose K15M | |
| | Sodium stearate fumarate | 5.00 |
| | Total | 100.00 |
| Immediate-release | Oseltamivir phosphate | 41.93 |
| layer | MCC 101QD | 44.00 |
| | Polyvinylpyrrolidone | 1.07 |
| | Croscarmellose sodium | 10.00 |
| | Sodium stearate fumarate | 3.00 |
| | Total | 100.00 |
| Isolation layer | Opadry 85F | 15.00 |
| | Purified water | 85.00 |
| | Total | 100.00 |

### Example 20

Formulation Form
180 mg oseltamivir sustained-release formulation

| | Component | Formulation ratio (%) |
|---|---|---|
| Sustained-release | Oseltamivir phosphate | 40.22 |
| layer | MCC 101QD | 24.78 |
| | Methacrylic acid-ethyl | 15.00 |
| | acrylate copolymer | |
| | L100-55 | |
| | Hydroxypropyl methyl | 15.00 |
| | cellulose K15M | |
| | Sodium stearate fumarate | 5.00 |
| | Total | 100.00 |
| Immediate-release | Oseltamivir phosphate | 41.93 |
| layer | MCC 101QD | 44.00 |
| | Polyvinylpyrrolidone | 1.07 |
| | Croscarmellose sodium | 10.00 |
| | Sodium stearate fumarate | 3.00 |
| | Total | 100.00 |
| Isolation layer | Opadry 85F | 15.00 |
| | Purified water | 85.00 |
| | Total | 100.00 |

### Example 21

Formulation Form
180 mg oseltamivir sustained-release formulation

| | Component | Formulation ratio (%) |
|---|---|---|
| Sustained-release | Oseltamivir phosphate | 32.85 |
| layer | MCC 101QD | 2.15 |
| | Methacrylic acid-ethyl | 40.00 |
| | acrylate copolymer | |
| | L100-55 | |
| | Hydroxypropyl methyl | 20.00 |
| | cellulose K15M | |
| | Sodium stearate fumarate | 5.00 |
| | Total | 100.00 |
| Immediate-release | Oseltamivir phosphate | 41.93 |
| layer | MCC 101QD | 44.00 |
| | Polyvinylpyrrolidone | 1.07 |
| | Croscarmellose sodium | 10.00 |
| | Sodium stearate fumarate | 3.00 |
| | Total | 100.00 |
| Isolation layer | Opadry 85F | 15.00 |
| | Purified water | 85.00 |
| | Total | 100.00 |

### Example 22

Formulation Form
180 mg oseltamivir sustained-release formulation

| | Component | Formulation ratio (%) |
|---|---|---|
| Sustained-release | Oseltamivir phosphate | 40.22 |
| layer | MCC 101QD | 29.78 |
| | Methacrylic acid-ethyl | 15.00 |
| | acrylate copolymer | |
| | L100-55 | |
| | Hydroxypropyl methyl | 10.00 |
| | cellulose K15M | |
| | Sodium stearate fumarate | 5.00 |
| | Total | 100.00 |
| Immediate-release | Oseltamivir phosphate | 41.93 |
| layer | MCC 101QD | 44.00 |
| | Polyvinylpyrrolidone | 1.07 |
| | Croscarmellose sodium | 10.00 |
| | Sodium stearate fumarate | 3.00 |
| | Total | 100.00 |
| Isolation layer | Opadry 85F | 15.00 |
| | Purified water | 85.00 |
| | Total | 100.00 |

### Example 23

Formulation Form
180 mg oseltamivir sustained-release formulation

| | Component | Formulation ratio (%) |
|---|---|---|
| Sustained-release | Oseltamivir phosphate | 40.22 |
| layer | MCC 101QD | 9.78 |
| | Methacrylic acid-ethyl | 30.00 |
| | acrylate copolymer | |
| | L100-55 | |
| | Hydroxypropyl methyl | 15.00 |
| | cellulose K4M | |
| | Sodium stearate fumarate | 5.00 |
| | Total | 100.00 |
| Immediate-release | Oseltamivir phosphate | 41.93 |
| layer | MCC 101QD | 44.00 |
| | Polyvinylpyrrolidone | 1.07 |
| | Croscarmellose sodium | 10.00 |
| | Sodium stearate fumarate | 3.00 |
| | Total | 100.00 |
| Isolation layer | Opadry 85F | 15.00 |
| | Purified water | 85.00 |
| | Total | 100.00 |

### Example 24

Formulation Form
180 mg oseltamivir sustained-release formulation

| | Component | Formulation ratio (%) |
|---|---|---|
| Sustained-release | Oseltamivir phosphate | 40.22 |
| layer | MCC 101QD | 19.78 |
| | Methacrylic acid-ethyl | 5.00 |
| | acrylate copolymer | |
| | L100-55 | |
| | Hydroxypropyl methyl | 10.00 |
| | cellulose K100M | |
| | Hydroxypropyl methyl | 20.00 |
| | cellulose K100 LV | |
| | Sodium stearate fumarate | 5.00 |
| | Total | 100.00 |
| Immediate-release | Oseltamivir phosphate | 41.93 |
| layer | MCC 101QD | 44.00 |
| | Polyvinylpyrrolidone | 1.07 |
| | Croscarmellose sodium | 10.00 |
| | Sodium stearate fumarate | 3.00 |
| | Total | 100.00 |
| Isolation layer | Opadry 85F | 15.00 |
| | Purified water | 85.00 |
| | Total | 100.00 |

### Example 25

Formulation Form
180 mg oseltamivir sustained-release formulation

| | Component | Formulation ratio (%) |
|---|---|---|
| Sustained-release | Oseltamivir phosphate | 40.22 |
| layer | MCC 101QD | 9.78 |
| | Methacrylic acid-ethyl | 30.00 |
| | acrylate copolymer | |
| | L100-55 | |
| | Hydroxypropyl methyl | 7.50 |
| | cellulose K100M | |
| | Hydroxypropyl methyl | 7.50 |
| | cellulose K15M | |
| | Sodium stearate fumarate | 5.00 |
| | Total | 100.00 |
| Immediate-release | Oseltamivir phosphate | 41.93 |
| layer | MCC 101QD | 44.00 |
| | Polyvinylpyrrolidone | 1.07 |
| | Croscarmellose sodium | 10.00 |
| | Sodium stearate fumarate | 3.00 |
| | Total | 100.00 |
| Isolation layer | Opadry 85F | 15.00 |
| | Purified water | 85.00 |
| | Total | 100.00 |

### Example 26

Formulation Form
180 mg oseltamivir sustained-release formulation

| | Component | Formulation ratio (%) |
|---|---|---|
| Sustained-release | Oseltamivir phosphate | 40.22 |
| layer | MCC 101QD | 9.78 |
| | Methacrylic acid-ethyl | 30.00 |
| | acrylate copolymer | |
| | L100-55 | |
| | Hydroxypropyl methyl | 15.00 |
| | cellulose K15M | |
| | Sodium stearate fumarate | 5.00 |
| | Total | 100.00 |
| Immediate-release | Oseltamivir phosphate | 26.27 |
| layer | MCC 101QD | 59.66 |
| | Polyvinylpyrrolidone | 1.07 |
| | Croscarmellose sodium | 10.00 |
| | Sodium stearate fumarate | 3.00 |
| | Total | 100.00 |
| Isolation layer | Opadry 85F | 15.00 |
| | Purified water | 85.00 |
| | Total | 100.00 |

### Preparation Method of Example 18 to Example 26

Preparation of sustained-release layer bulk granules: oseltamivir phosphate, microcrystalline cellulose and hydroxypropyl methyl cellulose were weighed and transferred to a granulator for premixing, purified water was used as granulation solution for wet granulation, and then wet granulated, fluidized bed drying, and dry granulated in sequence, then lubricant was added to obtain the sustained-release layer bulk granules.

Preparation of immediate-release layer bulk granules: oseltamivir phosphate and microcrystalline cellulose were weighed and transferred to a granulator for premixing, and polyvinylpyrrolidone-water was used as granulation solution for wet granulation (the amount of the final dried polyvinylpyrrolidone is shown in the formulation), and then wet granulated, fluidized bed drying, dry granulated in sequence, and then lubricant was added to obtain the immediate-release layer bulk granules.

Preparation of double-layer tablet: a double-layer tablet press was used to fill 150 mg of sustained-release layer bulk granules, then pre-pressed to obtain the sustained-release layer tablet core; then 30 mg of immediate-release layer granules were filled and pressed into tablet to obtain the immediate-release and sustained-release double-layered tablet core.

Isolation layer: an isolation layer coating solution was prepared and used to coat the above-mentioned immediate-release and sustained-release double-layered tablet core, and the percentage of coating weight gain in the tablet core was 2%.

TABLE 12
The weight ratio of the sustained-release material to the
total weight of the formulation in Example 18 to Example 26
Example 18 to Example 26

| | ratio of | ratio of | ratio of |
|---|---|---|---|
| | methacrylic acid- | hydroxypropyl | sustained- |
| | ethyl acrylate | methyl | release |
| | copolymer (%) | cellulose (%) | material (%) |
| Example 18 | 25 | 12 | 37 |
| Example 19 | 25 | 4 | 29 |
| Example 20 | 12 | 12 | 25 |
| Example 21 | 33 | 17 | 50 |
| Example 22 | 12 | 8 | 21 |
| Example 23 | 25 | 12 | 37 |
| Example 24 | 4 | 25 | 29 |
| Example 25 | 25 | 12 | 37 |
| Example 26 | 23 | 11 | 34 |
| Actual range | 4-33% | 4%-25% | 21-50% |
| The scope of the | 0%-35% | 4-25% | 3-50% |
| claims | | | |

Dissolution Data

In this example, the oseltamivir formulations prepared in Examples 18 to 26 were dissolved in a pH 6.8 medium, the volume of the medium was 900±9 mL, the temperature of the medium was 37.0±0.5° C., the paddle method was used, and the in vitro dissolution test was performed at 100 rpm/min. Time points for dissolution sampling in pH6.8: 0.25 h, 1 h, 2 h, 3 h, 4 h, 6 h, 8 h, 10 h, 12 h, 14 h and 16 h. Sampling location: from the top of the paddle to the midpoint of the liquid surface, 10 mm away from the inner wall of the dissolution vessel. In vitro dissolution content was tested by High Performance Liquid Chromatography (HPLC) method. Results are as shown below:

TABLE 13
Dissolution of the products of Example
18 to 26 in pH 6.8 medium (%) (n = 3)
Dissolution in pH 6.8 medium

| | 0.25 | 1 | 2 | 3 | 4 | 6 | 8 | 10 | 12 | 14 | 16 |
|---|---|---|---|---|---|---|---|---|---|---|---|
| Test number | h | h | h | h | h | h | h | h | h | h | h |
| Example 18 | 24 | 34 | 43 | 49 | 55 | 65 | 74 | 81 | 87 | 90 | 93 |
| Example 19 | 26 | 32 | 44 | 52 | 60 | 74 | 83 | 90 | 94 | 98 | — |
| Example 20 | 24 | 32 | 42 | 48 | 57 | 75 | 89 | 94 | 98 | 98 | — |
| Example 21 | 25 | 31 | 35 | 42 | 47 | 61 | 71 | 77 | 81 | 85 | 87 |
| Example 22 | 26 | 35 | 45 | 53 | 61 | 78 | 89 | 95 | 98 | 99 | — |
| Example 23 | 27 | 35 | 47 | 51 | 57 | 72 | 81 | 88 | 93 | 97 | — |
| Example 24 | 22 | 31 | 38 | 44 | 49 | 58 | 66 | 72 | 76 | 80 | 83 |
| Example 25 | 24 | 32 | 41 | 46 | 52 | 63 | 73 | 78 | 82 | 87 | 89 |
| Example 26 | 24 | 34 | 42 | 49 | 55 | 65 | 74 | 81 | 87 | 90 | 93 |

Dissolution Data

In this example, the oseltamivir formulations prepared in Examples 18 to 26 were acid resistant in 0.1M HCl for 1 h and then transferred to a pH 6.8 medium for dissolution. The volume of the medium was 900±9 mL, and the temperature of the medium was 37.0±0.5° C., the paddle method was used, and the in vitro dissolution test was performed at 75 rpm/min. Time points for dissolution sampling in 0.1M HCl: 0.25 h, 1 h, time points for dissolution sampling in pH 6.8: 1 h, 2 h, 3 h, 4 h, 6 h, 8 h, 10 h, 12 h, 14 h, 16 h. Sampling location: from the top of the paddle to the midpoint of the liquid surface, 10 mm away from the inner wall of the dissolution vessel. In vitro dissolution content was tested by High Performance Liquid Chromatography (HPLC) method. Results are as shown below:

TABLE 14
Dissolution of the products from Example 18 to 26 acid resistant
in 0.1M HCl for 1 h and then transferred to pH 6.8 medium (%) (n = 3)
Dissolution of acid resistance in 0.1M HCl for 1 h and then transferred
to pH 6.8 medium (2 media dissolution times were accumulated)

| | 0.25 | 1 | 2 | 3 | 4 | 5 | 7 | 9 | 11 | 13 | 15 |
|---|---|---|---|---|---|---|---|---|---|---|---|
| Test number | h | h | h | h | h | h | h | h | h | h | h |
| Example 18 | 25 | 33 | 38 | 42 | 48 | 54 | 64 | 74 | 82 | 88 | 92 |
| Example 19 | 27 | 35 | 42 | 50 | 57 | 64 | 79 | 88 | 91 | 95 | 98 |
| Example 20 | 25 | 34 | 37 | 43 | 50 | 59 | 77 | 87 | 95 | 99 | 101 |
| Example 21 | 24 | 31 | 36 | 41 | 46 | 51 | 61 | 71 | 77 | 81 | 87 |
| Example 22 | 26 | 35 | 40 | 46 | 54 | 62 | 78 | 89 | 95 | 98 | 99 |
| Example 23 | 25 | 34 | 40 | 46 | 51 | 57 | 69 | 78 | 85 | 89 | 92 |
| Example 24 | 24 | 32 | 36 | 41 | 46 | 52 | 63 | 74 | 82 | 89 | 94 |
| Example 25 | 23 | 34 | 37 | 45 | 51 | 57 | 69 | 78 | 85 | 91 | 93 |
| Example 26 | 24 | 33 | 38 | 42 | 47 | 52 | 63 | 72 | 79 | 86 | 91 |

Reference throughout this specification to “an embodiment,” “some embodiments,” “one embodiment”, “another example,” “an example,” “a specific example,” or “some examples,” means that a particular feature, structure, material, or characteristic described in connection with the embodiment or example is included in at least one embodiment or example of the present disclosure. In this specification, schematic representations of the above phrases are not necessarily referring to the same embodiment or example. Furthermore, the particular features, structures, materials, or characteristics may be combined in any suitable manner in one or more embodiments or examples. In addition, those skilled in the art can integrate and combine different embodiments, examples or the features of them as long as they are not contradictory to one another.

Although explanatory embodiments have been shown and described, it would be appreciated by those skilled in the art that the above embodiments cannot be construed to limit the present disclosure, and changes, alternatives, and modifications can be made in the embodiments without departing from spirit, principles and scope of the present disclosure.

1-43. (canceled)

44. An oseltamivir formulation comprising oseltamivir or a salt thereof, the formulation being effective at a once-daily dosage to a patient in need thereof.

45. The oseltamivir formulation of claim 44, wherein at least one of:
(i) a release amount of oseltamivir for any 1 h time period of ≤4 h is 25%-55%;
(ii) a release amount of oseltamivir in the first hour is 25%-55%;
(iii) a release amount of oseltamivir at 4 h is 25%-90%; (iv) a release amount of oseltamivir at 10 h is greater than 70%; and
(v) a release amount of oseltamivir at 10 h is 70%-99%.

46. The oseltamivir formulation of claim 44, wherein at least one of:
(i) a weight ratio of oseltamivir is 3%-50% of a total weight of the formulation;
(ii) after administration of a once-daily dose, a peak-to-valley ratio of a plasma concentration in vivo of an oseltamivir active metabolite within 24 h is less than 2.5:1; and
(iii) the oseltamivir formulation has a single-dose strength of 60 mg-300 mg by weight of oseltamivir.

47. The oseltamivir formulation of claim 44, further comprising at least one sustained-release material,
wherein at least one of:
(i) a weight ratio of the sustained-release material is 3%-50% of a total weight of the formulation; or
(ii) the sustained-release material includes at least one selected from the group consisting of ethyl cellulose, hydroxypropyl methyl cellulose, cellulose acetate, poly oxyethylene, polyvinyl alcohol, methacrylic acid-ethyl acrylate copolymer RL, methacrylic acid-ethyl acrylate copolymer RS, methacrylic acid-ethyl acrylate copolymer NE 30D, methacrylic acid-ethyl acrylate copolymer L100-55, hypromellose acetate succinate, glyceryl behenate, chitosan, carbomer, carnauba wax, a 30% dispersion of polyvinyl acetate, a blend of polyvinyl acetate and povidone, cellulose acetate titanate, sodium alginate, sodium carboxymethyl cellulose, carrageenan, cetyl alcohol, stearyl alcohol, cetostearyl alcohol, and paraffin.

48. The oseltamivir formulation of claim 44, further comprising at least one sustained-release material,
wherein a weight ratio of oseltamivir is 3%-50% of a total weight of the formulation, and
a release amount of oseltamivir at 4 h is 25%-90%, and
a release amount of oseltamivir at 10 h is greater than 70%.

49. The oseltamivir formulation of claim 44, further comprising a sustained-release part containing oseltamivir or salt thereof and an immediate-release part containing oseltamivir or salt thereof.

50. The oseltamivir formulation of claim 49, wherein, in a single-dose, an oseltamivir strength in the immediate-release part is 10 mg-75 mg, and an oseltamivir strength in the sustained-release part is 30 mg-225 mg.

51. The oseltamivir formulation of claim 49, wherein the oseltamivir formulation is effective for treatment of influenza A or influenza B in adults, after administration of a once-daily dose,
a plasma concentration in vivo of an oseltamivir active metabolite within 24 h is greater than 100 ng/mL; and the oseltamivir formulation has a single-dose strength of 150 mg-300 mg by weight of oseltamivir.

52. The oseltamivir formulation of claim 49, wherein the oseltamivir formulation is effective for treatment of influenza A or influenza B in children, after administration of a once-daily dose,
a plasma concentration in vivo of an oseltamivir active metabolite within 24 h is greater than 100 ng/mL; and
the oseltamivir formulation has a single-dose strength of 60 mg-180 mg by weight of oseltamivir.

53. The oseltamivir formulation of claim 49, wherein after administration of a once-daily dose, at least one of (i) a plasma concentration in vivo of an oseltamivir active metabolite within 2 h is more than 20% of Cmax, and a plasma concentration in vivo within 24 h is more than 30% of Cmax, and (ii) the oseltamivir formulation continuously releases oseltamivir for a period of at least 24 h.

54. The oseltamivir formulation of claim 49, wherein the plasma concentration in vivo of an oseltamivir active metabolite is greater than 100 ng/mL, and a maintenance time is greater than 16 h.

55. The oseltamivir formulation of claim 44, wherein the oseltamivir formulation is a biphasic release formulation comprising a sustained-release part containing oseltamivir or salt thereof and an immediate-release part containing oseltamivir or salt thereof.

56. The oseltamivir formulation of claim 55, wherein, by total weight of oseltamivir in the formulation, at least one of (i) a weight ratio of oseltamivir in the immediate-release part is 20%-50%, and (ii) a weight ratio of oseltamivir in the immediate-release part and in the sustained-release part is 1:4-1:1.

57. The oseltamivir formulation of claim 55, further comprising a sustained-release material, the sustained-release material comprising at least one of hydroxypropyl methyl cellulose and methacrylic acid-ethyl acrylate copolymer,
wherein a weight ratio of the sustained-release material is 3%-50% of a total weight of the formulation,
a weight ratio of hydroxypropyl methyl cellulose is 4%-30% of a total weight of the formulation, and
a weight ratio of methacrylic acid-ethyl acrylate copolymer is 0-35% a total weight of the formulation.

58. The oseltamivir formulation of claim 44, wherein a release amount of oseltamivir at 1 h is 25%-55%, a release amount of oseltamivir at 4 h is 25%-90%, a release amount of oseltamivir at 10 h is greater than 70%, and a weight ratio of oseltamivir is 3%-50% a total weight of the formulation, and
the formulation further comprises at least one sustained-release material, a weight ratio of the sustained-release material is 3%-50% of a total weight of the formulation,
after administration of a once-daily dose, a peak-to-valley ratio of a plasma concentration in vivo of an oseltamivir active metabolite within 24 h is less than 2.5:1,
the oseltamivir formulation is effective for treatment of influenza A or influenza B in adults or children, and
after administration of the once-daily dose, a plasma concentration in vivo of the oseltamivir active metabolite within 24 h is greater than 100 ng/mL, a plasma concentration in vivo of the oseltamivir active metabolite at 2 h is more than 20% of Cmax, and a plasma concentration in vivo at 24 h is more than 30% of Cmax.

59. A tablet, a capsule, or a suspension comprising the formulation of claim 44.

60. The oseltamivir formulation of claim 44, further comprising a sustained-release part and an immediate-release part, the sustained-release part comprising a first bead, and the immediate-release part comprising a second bead,
wherein:
(i) the first bead is prepared by uniformly mixing oseltamivir and a matrix sustained-release material, and the second bead is prepared by uniformly mixing oseltamivir and a matrix material,
(ii) the first bead contains oseltamivir and an exterior that is coated with a sustained-release coating layer formed by high polymer and pharmaceutical excipients, and the second bead contains oseltamivir and an exterior that is coated with an immediate-release coating layer formed by a water-soluble polymer film-forming material and a plasticizer,
(iii) the first bead is prepared by an extrusion spheronization method after mixing oseltamivir with the matrix sustained-release material, and the second bead is prepared by the extrusion spheronization method after mixing oseltamivir with the pharmaceutical excipients, or
(iv) the first bead is prepared by spraying oseltamivir and a sustained-release film-forming material on a blank pellet, and the second bead is prepared by spraying oseltamivir and an immediate-release coating material on a blank pellet.

61. The oseltamivir formulation of claim 60, wherein (i) the first bead and the second bead are mixed and compressed into a double-layer or multi-layer tablet, (ii) the first bead and the second bead are mixed and directly filled in capsules, or (iii) the first bead and the second bead are mixed and then bagged.

62. A preparation method of the oseltamivir formulation of claim 60, the method comprising one process selected from the following groups of processes:
Group A:
(1) obtaining the immediate-release part by wrapping the immediate-release coating layer formed by the water-soluble polymer film-forming material, the plasticizer and oseltamivir around a core of the blank pellet;
(2) obtaining the sustained-release part by wrapping the sustained-release coating layer formed by the high polymer and the pharmaceutical excipients around the immediate-release part obtained in step (1); and
(3) filling the immediate-release part obtained in step (1) and the sustained-release part obtained in step (2) into capsules, or compressing into tablets, or bagging to obtain the oseltamivir formulation;
Group B:
(1) obtaining the sustained-release part by wrapping the sustained-release coating layer formed by the high polymer and the other pharmaceutical excipients around the first bead containing oseltamivir; and
(2) obtaining the oseltamivir formulation by wrapping the immediate-release coating layer formed by the water-soluble polymer film-forming material, the plasticizer and oseltamivir around the sustained-release part obtained in step (1);
Group C:
(1) obtaining the sustained-release part by wrapping the sustained-release coating layer formed by oseltamivir, the high polymer and the pharmaceutical excipients around the core of the blank pellet; and
(2) obtaining the oseltamivir formulation by wrapping the immediate-release coating layer formed by the water-soluble polymer film-forming material, the plasticizer and oseltamivir around the sustained-release part obtained in step (1); and
Group D:
obtaining the oseltamivir formulation by wrapping the immediate-release coating layer formed by the water-soluble polymer film-forming material, the plasticizer and oseltamivir around the sustained-release part comprising the first bead containing oseltamivir and the matrix sustained-release material.

63. The preparation method of claim 62, wherein at least one of:
(i) the high polymer comprises at least one selected from the group consisting of ethyl cellulose, cellulose acetate, methacrylic acid-ethyl acrylate copolymer RL, methacrylic acid-ethyl acrylate copolymer RS, methacrylic acid-ethyl acrylate copolymer NE 30D, methacrylic acid-ethyl acrylate copolymer L100-55, hypromellose acetate succinate, carnauba wax, a 30% dispersion of polyvinyl acetate, and a blend of polyvinyl acetate and povidone,
(ii) the pharmaceutical excipients comprise one selected from the group consisting of plasticizers, anti-sticking agents, emulsifiers, and porogens;
(iii) the water-soluble polymer film-forming material comprises at least one selected from the group consisting of hydroxypropyl methyl cellulose, povidone, polyvinyl alcohol, and hydroxypropyl cellulose,
(iv) the matrix sustained-release material comprises at least one selected from group consisting of hydroxypropyl methyl cellulose, polyoxyethylene, polyvinyl alcohol, ethyl cellulose, glyceryl behenate, chitosan, carbomer, sodium alginate, sodium carboxymethyl cellulose, carrageenan, cetyl alcohol, stearyl alcohol, cetostearyl alcohol, and paraffin, and
(v) the immediate-release coating layer is externally wrapped with an isolation layer, and the isolation layer comprises at least one selected from the group consisting of a water-soluble polymer film-forming material, a plasticizer, an anti-sticking agent, and an opacifier.

64. The tablet, the capsule, or the suspension of claim 59, wherein the tablet is a double-layer tablet, one layer of the double-layer tablet comprising an immediate-release part and the other layer comprising a sustained-release part, and
the sustained-release part forms a tablet core, and the immediate-release part is wrapped around the tablet core.
